(19) United States
(12) Patent Application Publication
(10) Pub. No.: US 2011/0177091 A1
(43) Pub. Date: Jul. 21, 2011

(54) Inhibition of Tumor Angiogenesis by Inhibition of Peroxiredoxin 1 (PRX1)

(76) Inventors: Sandra O. Gollnick, Williamsville, NY (US); Jonah Riddell, Buffalo, NY (US)

(21) Appl. No.: 12/963,039

(22) Filed: Dec. 8, 2010

Related U.S. Application Data
(60) Provisional application No. 61/267,656, filed on Dec. 8, 2009.

(51) Int. Cl. A61K 39/395; A61P 35/00

(52) U.S. Cl. 424/155.1; 424/174.1

(57) ABSTRACT

Provided is a method for inhibiting angiogenesis in a tumor. The method involves administering to an individual who has a tumor a composition that contains an agent capable of inhibiting binding of peroxiredoxin 1 (Prx1) to Toll like receptor 4 (TLR4) such that angiogenesis in the tumor is inhibited subsequent to the administration. The agent that is capable of inhibiting binding of Prx1 to TLR4 can be an antibody to Prx1, a Prx1 binding fragment of the antibody, or a peptide. The peptide can be capable of inhibiting the formation of a Prx1 decamer, or can inhibit binding of Prx1 to TLR4 by steric interference, or by competitions with Prx1 for TLR4 binding. The tumor in which angiognesis is inhibited can be any type of cancer tumor.

### CROSS-REFERENCE TO RELATED APPLICATION

This application claims priority to U.S. application No. 61/267,656, filed on Dec. 8, 2009, the disclosure of which is incorporated herein by reference.

### FIELD OF THE INVENTION

The present invention is related generally to the field of tumor therapy, and more specifically related to inhibition of angiogenesis in a tumor by inhibition of Prx1 binding to Toll-like receptor 4 (TLR4).

### BACKGROUND OF THE INVENTION

Prx1 is a member of the typical 2-cysteine peroxiredoxin family, whose major intracellular functions are as a regulator of hydrogen peroxide signaling through its peroxidase activity and as a protein chaperone. Prx1 expression is elevated in various cancers, including esophageal, pancreatic, lung, follicular thyroid, and oral cancer. Elevated Prx1 levels have been linked with poor clinical outcomes and diminished overall patient survival. Recent studies have demonstrated that Prx1 can be secreted by non-small cell lung cancer cells, possibly via a non-classical secretory pathway. To date, the function of secreted Prx1 is unknown. There is an ongoing and unmet need to develop therapies for tumors that express Prx1.

### SUMMARY OF THE INVENTION

The present invention provides a method for inhibiting angiogenesis in a tumor. The method comprises administering to an individual who has a tumor a composition comprising an agent capable of inhibiting binding of peroxiredoxin 1 (Prx1) to Toll like receptor 4 (TLR4) such that angiogenesis in the tumor is inhibited subsequent to the administration. In one embodiment, the agent that is capable of inhibiting binding of Prx1 to TLR4 is an antibody to Prx1, or a Prx1 binding fragment of the antibody. In another embodiment, the agent that is capable of inhibiting binding of Prx1 to TLR4 is a peptide. In one embodiment, the peptide is a fragment of Prx1. The peptide can be capable of, for example, inhibiting the formation of a Prx1 decamer, or can inhibit binding of Prx1 to TLR4 by steric interference, or by competitions with Prx1 for TLR4 binding.

The individual treated by the method of the invention can be an individual who is in need of treatment for any tumor. In particular non-limiting embodiments, the tumor is selected from prostate, thyroid, lung, bladder breast and oral cancer tumors.

Inhibition of angiogenesis can comprise a change in any indicator of a reduction of angiogenesis known to those skilled in the art. In various non-limiting embodiments, the inhibition of angiogenesis can comprise a reduction in number or size of blood vessels in the tumor, and/or an increase in permeability of blood vessels in the tumor. Further, inhibiting angiogenesis can be correlated with a reduction in vascular endothelial growth factor (VEGF) mRNA, VEGF protein, or a combination thereof in the tumor.

### BRIEF DESCRIPTION OF THE FIGURES

FIG. 1. Prx1 stimulates cytokine secretion from macrophages. (A) TG-elicited macrophages were analyzed by flow cytometry for expression of CD11b, Gr1, and F4/80. A representative histogram of 3 independent isolations is shown and depicts Gr1 and F4/80 expression by CD11b⁺ cells. Numbers in the insets indicate the percentages of CD11b⁺ cells in each quadrant. (B) TG-elicited macrophages were incubated with stimulants for 24 h; supernatants were harvested and analyzed for TNF-α (open bars) and IL-6 levels (gray bars). Results are shown as pg/ml and are representative of three independent experiments; error bars represent standard deviation. (C) TG-elicited macrophages were incubated for 24 h with media only (black bars), 100 nM LPS or 2000 nM Prx1 (open bars), 100 nM LPS or 2000 nM Prx1 pre-incubated for 20 minutes with 10 ug/mL polymyxin B (hatched bars), or 100 nM LPS or denatured 2000 nM Prx1 (gray bars). Asterisks indicate P≦0.01 as compared to cells treated with Prx1 or LPS alone. (D) TG-elicited macrophages were incubated with media alone, Prx1 (50 nM) or LPS (100 nM) for 24 h in the presence (gray bars) or absence (open bars) of 10% FBS. Supernatants were harvested and analyzed for IL-6 levels. Results are shown as pg/ml; error bars represent standard deviation.

FIG. 2. Prx1 stimulates dendritic cell maturation and activation. Immature bone marrow derived dendritic cells (iBMDCs) were incubated with media alone, 20-200 nM Prx1 or 100 nM LPS for 24 h. (A) Following incubation cells were analyzed by flow cytometry for expression of CD11c and CD86. Results are shown as percent total cells; error bars represent standard deviation. (B) Supernatants were harvested and analyzed for TNF-α. Results are shown as pg/ml and are representative of three independent experiments; error bars represent standard deviation. (C) TG-elicited macrophages were incubated with media harvested from prostate tumor cell lines that were transfected with cDNA encoding for either control shRNA (Scramble) or shRNA specific for Prx1 (shPrx1) or in media harvested from cells expressing Prx1 specific shRNA to which 50 nM exogenous Prx1 had been added (shPrx1+Prx1). Following 24 h incubation, supernatants were harvested and analyzed for TNF-α. Results are shown as pg/ml and are representative of three independent experiments; error bars represent standard deviation. **: P≦0.01 when compared to TNF-α levels secreted by cells incubated with media alone; ##: P≦0.01 when compared to TNF-α levels secreted by cells incubated with media from cells expressing control shRNA; ††: P≦0.01 when compared to TNF-α levels secreted by cells incubated with media from cells expressing shRNA specific for Prx1.

FIG. 3. Prx1 induced cytokine secretion is TLR4 dependent. (A) iBMDCs were isolated from C57BL/6 (TLR4^{+/+}; open bars) and C57BL/10ScNJ (TLR4^{-/-}; closed bars) mice and stimulated with 200 nM Prx1, 100 nM LPS, or 100 mM Pam₃Cys. Supernatants were collected and analyzed by IL-6 ELISA kits. (B) TG-elicited macrophages were isolated from C57BL/6 (TLR4^{+/+}; open bars) and C57BL/10ScNJ (TLR4^{-/-}; closed bars) mice and stimulated with 200 nM Prx1, 100 nM LPS, or 100 mM Pam₃Cys. Supernatants were collected and analyzed by IL-6 ELISA kits. Results are presented as pg/ml; error bars represent standard deviation; asterisks indicate P values less that 0.01. (C) Naïve C57BL/6 (TLR4^{+/+}; open bars) and C57BL/10ScNJ (TLR4^{-/-}; closed bars) mice were injected i.p. with 200 nm Prx1. Six hours later, blood was collected and analyzed by ELISA for the presence of IL-6. Results are presented as pg/ml; error bars represent standard deviation; asterisks indicate P≦0.0002.

FIG. 4: Interaction of Prx1 with TLR4 is dependent upon CD14 and MD2 (A) TG-elicited macrophages were isolated from C57BL/6 mice and stimulated with 50 nM Prx1 in the presence or absence of control or blocking antibodies to Prx1, CD14 or MD2 for 24 h. Supernatants were collected and analyzed by IL-6 ELISA kits. Results are presented as pg/ml; error bars represent SEM; asterisks indicate P values less that 0.01. (B) TG-elicited macrophages were harvested and cell lysates were precipitated with antibodies to TLR4, TLR2, and mouse/goat IgG as described in Materials and Methods; resulting precipitates were separated by SDS-PAGE and probed by Western blot analysis for the presence of Prx1. Blots were also probed with antibodies to TLR4 or TLR2 as a loading control. (C) TG-elicited macrophages were harvested and cell lysates were incubated with antibodies to TLR4 or mouse/goat IgG as described in Materials and Methods; resulting precipitates were separated by SDS-PAGE and probed by Western blot analysis for the presence of Prx1, CD14 and MD2. Blots were also probed with antibodies to TLR4 as a loading control.

FIG. 5: Kinetics of TLR4/Prx1 Interaction. (A) TG-elicited macrophages were stimulated with 200 nM FITC-Prx1 or PE-conjugated anti-TLR4 (PE-TLR4). Samples were harvested at the indicated times samples and cell populations were analyzed by Amnis technology. Representative examples of immunostained cells and a merged image of the two stains for each time point are shown. The far right column shows a histogram of the pixel by pixel statistical analysis of each cell (n=5,000) analyzed in which the y-axis is number of cells and the x-axis is the similarity coefficient between Prx1 and TLR4. (B) The average similarity coefficient of all cells for each time point is shown; error bars represent standard deviation.

FIG. 6. Prx1 Binding to TLR4 is Structure Dependent (A) TG-elicited macrophages isolated from TLR4^{+/+} (white bars) or TLR4^{-/-} macrophages (filled bars) and incubated with media (None), Prx1, Prx1C52S, or Prx1C83S at 200 nM for 24 h and supernatants were harvested and analyzed for the presence of TNF-α and IL-6. (B) TG-elicited macrophages isolated from TLR4^{+/+} (white bars) or TLR4^{-/-} macrophages (filled bars) and incubated with 2000 nM of FITC-labeled proteins for 20 minutes, followed by analysis by flow cytometry. Viable cells were selected for analysis by elimination of 7-AAD high populations. Results were normalized for any differences in FITC-labeling and reported in MFI/FITC per nM protein; error bars represent standard deviation. Asterisks indicate a P value≦0.01. (C) TG-elicited macrophages were incubated with FITC-BSA (squares), Prx1 (dark circles), Prx1C52S (gray circles), and Prx1 C83S (open circles) at various concentrations for 20 min and analyzed by flow cytometry. Results are normalized for differences in FITC-labeling and reported in MFI/FITC per nM protein. Each curve is representative of three individual trials. (D) TG-elicited macrophages were incubated with 1000 nM Prx1, washed and incubated with increasing concentrations of competitors: OVA (squares), Prx1 (dark circles), Prx1C52S (gray circles), Prx1C83S (open circles). Results are shown as a percentage MFI of FITC-Prx1 with no competitor; error bars represent standard deviation. All experiments were performed in triplicate and the combined results are presented.

FIG. 7. Prx1 stimulation of macrophages is MyD88 dependent and leads nuclear translocation of NFκB. (A) Stable transfectants of the RAW264.7 macrophage cell line containing control (open bar) or MyD88 DN (filled bars) expressing plasmids were stimulated with 100 nM LPS or 1000 nM Prx1 for 24 h and the resulting supernatants were assayed for IL-6 expression by ELISA. ELISA analysis was performed in three independent experiments; error bars represent standard deviation. Asterisks indicate a P value≦0.001. (B) TG-elicited macrophages isolated from C3H/HeNCr (TLR4^{+/+}) and C3H/HeNJ (TLR4^{-/-}) mice were stimulated with 200 nM Prx1 in complete media. At the indicated time points cells were stained with FITC conjugated antibodies to NFκB p65 and DRAQ5 (nuclear stain) for 10 min and analyzed using Amnis technology. The furthest right column shows a pixel by pixel statistical analysis of the similarity of NFκB and nuclear staining (C) The average numerical value of the overall similarity coefficients for each time point in both C3H/HeNCr (filled circles) and C3H/HeNJ (open circles) macrophages is; error bars represent standard deviation. (D) TG-elicited macrophages were incubated with the indicated concentrations of Prx1 for 1 hour. EMSA analysis was performed as described in Example 1.

FIG. 8. Expression of shRNA specific for Prx1 in PC-3M cells leads to a decrease in Prx1 expression. (A) Cell lysate isolated from PC-3M cells (right panel) engineered to express control (Scramble) shRNA or Prx1 specific shRNA (shPrx1) was separated by gel electrophoresis, blotted and probed with antibodies specific for Prx1. (B) Expression of shRNA specific for Prx1 leads to decreased Prx1 levels. PC3-M cell lines engineered to express either control shRNA (Scramble) or shRNA specific for Prx1′ were harvested and analyzed for expression of Prx1 or Prx2 by Western analysis. (C) Prx1 stimulation of IL-6 secretion from TG-elicited macrophages is dependent upon CD14 and MD2, which are cofactors of TLR4. TG-elicited macrophages were isolated from C57BL/6 mice and stimulated with LPS in the presence or absence of control or blocking antibodies to CD14 or MD2 for 24 h. Supernatants were collected and analyzed by IL-6 ELISA kits. Results are presented as pg/ml; error bars represent SEM.

FIG. 9. Prx1 Expression in CaP. Prostate cancer (CaP) tissue microarrays containing biopsies from 163 patients and normal tissue were analyzed for Prx1 expression by immunohistochemistry using a monoclonal antibody specific for Prx1. (A) Representative sections from benign/normal patient and a CaP patient are shown. (B) Prx1 expression was quantified by densitometry and divided into tiers based on expression level. Tumor grade was determined by a clinical pathologist. Results are plotted as mean expression vs. grade; error bars represent SD and * indicate P<0.05.

FIG. 10. Prx1 expression controls CaP growth. PC-3M, a human prostate tumor cell line, or C2H, a murine tumor cell line, cells were engineered to express control shRNA (Scramble) or shRNA specific for Prx1 (shPrx1) in the presence or absence of shRNA resistant Prx1 (shPrx1+sRP). Cells were implanted into SCID (A) or C57BL/6 (TLR4+/+) or TLR4−/− mice (B) and tumor growth was monitored for at least 60 days or until tumors reached 400 mm3. Lower panels demonstrate the level of Prx1 expression in tumors.

FIG. 11. Inhibition of Prx1 Expression Does Not Effect Cell Growth In Vitro or Cell Death In Vivo (A) Growth of PC-3M and C2H prostate cells engineered to express control (scramble) or Prx1 specific shRNA (shPrx1) was determined by clonogenic assay. (B) PC-3M CaP tumors expressing control (scramble) or Prx1 specific shRNA (shPrx1) grown in SCID mice were harvested and examined for expression of caspase 3 by immunohistochemistry. Representative sections are shown above and densitometry quantization is shown below.

FIG. 12. Prx1 Expression Affects Tumor Vasculature. PC-3M tumors expressing control (scramble) or Prx1 specific shRNA (shPrx1) were harvested when they had reached 100 mm3 in size and analyzed for expression of Prx1 and CD31 (a marker of vascular endothelial cells). (A) Representative sections are shown. (B-D) Expression was quantified by densitometry. Each symbol represents a separate tumor. A total of 26 fields were examined/tumor and the results were averaged to give the expression/tumor. Lines indicate the mean; **=P<0.01.

FIG. 13 Prx1 regulates tumor vasculature function. Vascular function is dependent upon association of endothelial cells (CD31⁺) and pericytes (NG2⁺). Scramble and shPrx1 PC-3M tumor sections from 150 mm³ tumors were stained with antibodies specific for CD31 or NG2. Representative individual or merged images from 5 sets of tumors are shown. These results indicate that Prx1 expression regulates pericyte association with endothelial cells.

FIG. 14. Prx1 Expression Affects Vascular Function. PC-3M tumors expressing control (scramble) or Prx1 specific shRNA (shPrx1) were harvested when they had reached 100 mm3 in size and analyzed by MRI for vascular permeability as determined by permeability of a contrast agent (change in relaxation rate/min). Representative images are were obtained 10 and 45 minutes post-injection of the contrast agent and quantification of the images is shown in the graph. Error bars represent SD; n=5 tumors/group.

FIG. 15. Prx1 Expression Effects VEGF Expression by Tumor and Host Cells. PC-3M tumors expressing control (scramble) or Prx1 specific shRNA (shPrx1) were harvested when they had reached 100 mm3 in size. Tumors were minced and tumor lysate was prepared by homogenization. (A) Human VEGF (hVEGF), derived from the tumor cells, and (B) murine mVEGF, derived from the host cells, levels were determined by ELISA. Results are expressed as pg/μg of total protein. Each symbol represents a separate tumor.

FIG. 16. VEGF Induction is Dependent Upon TLR Expression. Thioglycollate-elicited macrophages were isolated from TLR4+\+ or TLR4−\− mice and incubated with media, recombinant Prx1 (20 nM), LPS (100 ng/mL), a TLR4 agonist, or P3C (20 nM), a TLR2 agonist for 24 h. Supernatant was collected and the level of VEGF expression was determined by ELISA. Each assay contained three replicates/condition and the experiment was repeated a minimum of twice. Error bars represent SD; *=P<0.05

FIG. 17. Prx1 Induction of VEGF Promoter Activity is TLR Dependent. PC-3M cells expressing control (scramble) or Prx1 specific shRNA (shPrx1) were transfected with a reporter plasmid in which firefly luciferase expression was driven by the murine VEGF promoter and a reporter plasmid in which Renilla luciferase expression was driven by a CMV promoter in the presence or absence of a plasmid expressing a dominant/negative MyD88 protein, which inhibits TLR4 signal transduction. Cells were incubated with increasing amounts of Prx1; luciferase activity was determined after 24 h. Each assay contained three replicates/condition and the experiment was repeated a minimum of twice. Error bars represent SD; *=P<0.05, **=P<0.01.

FIG. 18. Prx1 Induced Endothelial Cell Migration is TLR4 Dependent. (A) Matrigel was infused with recombinant Prx1 and injected s.c. into C57BL/6 mice; 14 days following injection, mice were euthanized and matrigel plugs were recoved. The amount of hemoglobin/mg of matrigel was determined as an indication of endothelial cell migration. (B) Parental HUVEC endothelial cells or HUVEC cells expressing a dominant/negative mutant of MyD88 were placed in the upper chamber a transwell; collagen infused with culture media harvested from PC-3M cells expressing control shRNA (scramble) or shRNA specific for Prx1 (shPrx1) was placed in the lower chamber. The number of migrating endothelial cells was determined optically after 24 h of incubation. Results are expressed as cells/transwell. Each assay contained three replicates/condition and the experiment was repeated a minimum of twice. Error bars represent SD; **=P<0.01

FIG. 19. Prx1 Induced Endothelial Cell Proliferation is TLR4 Dependent. Parental HUVEC endothelial cells or HUVEC cells expressing a dominant/negative mutant of MyD88 were incubated with culture media (1640), conditioned media harvested from PC-3M cells expressing control shRNA (scramble) or shRNA specific for Prx1 (shPrx1); the number of cells was determined by trypan blue staining after 24 h of incubation. Results are expressed as percent proliferation with proliferation observed by cells incubated in culture media set at 100%. Each assay contained three replicates/condition and the experiment was repeated a minimum of twice. Error bars represent SD; **=P<0.01.

FIG. 20. Prx1 Induced Endothelial Cell Proliferation is Dependent Upon Chaperone Activity. HUVEC endothelial cells were incubated with PBS, recombinant Prx1 (rPrx1), a Prx1 mutant lacking peroxidase activity (rC52S) or a Prx1 mutant lacking chaperone activity (rC83S; all at 20 nM); the number of cells was determined by trypan blue staining after 24 h of incubation. Results are expressed as fold proliferation with proliferation observed by cells incubated with PBS being set a 1. Each assay contained three replicates/condition and the experiment was repeated a minimum of twice. Error bars represent SD; **=P<0.01.

FIG. 21. Prx1 Induced Endothelial Cell Proliferation is TLR4 Dependent. HUVEC endothelial cells were incubated with culture media (1640) or conditioned media harvested from PC-3M cells expressing shRNA specific for Prx1 in the presence of control (IgG) antibodies or antibodies specific for Prx1 or VEGF; proliferation was determined by MTT assay after 24 h of incubation. Each assay contained three replicates/condition and the experiment was repeated a minimum of twice. Error bars represent SD. Antibodies specific for Prx1 were obtained from Lab Frontier (Seoul, South Korea); this antibody is specific for Prx1 and detects only a single band in Western analysis of cells that express Prx1 (FIG. 8).

FIG. 22 provides a representation of data showing that Prx1 regulates VEGF protein and mRNA production.

FIG. 23 provides a representation of data showing that Prx1 regulates VEGF promoter activity.

FIG. 24 provides a representation of data illustrating that Prx1 control of the VEGF promoter is dependent upon the HIF-α binding element.

FIG. 25 provides a representation of data illustrating that Prx1 stimulation of HIF-α activity is MyD88 and NF-κB dependent.

### DESCRIPTION OF THE INVENTION

The present invention is based on the unexpected discovery that Peroxiredoxin 1 (Prx1) is a ligand for Toll-like receptor 4 (TLR4), and that inhibition of its interaction with TLR4 can be exploited for inhibition of angiogenesis.

In the present invention we have demonstrated that disrupting Prx1 binding and/or activation of TLR4 by Prx1 can inhibit angiogenesis, and in particular, can inhibit angiogenesis in tumors. Thus, the invention provides in one embodiment a method for inhibiting angiogenesis in a tumor. The method comprises administering to an individual a composition comprising an agent capable of disrupting Prx1 binding and/or activation of TLR4, such that angiogenesis in a tumor is reduced. The method of the invention is accordingly suitable for inhibiting the growth of a tumor, wherein in one embodiment, inhibition of growth of a tumor is measured by reducing tumor volume or by inhibiting an increase in tumor volume. The individual to whom the composition is administered can be an individual diagnosed with, suspected of having, or at risk for developing a tumor.

The amino acid sequence of Prx1 and DNA and RNA sequences encoding it are well known in the art, and it is expected that the invention will function by inhibiting TLR4 binding of Prx1 expressed in any individual, including any splice/variant and/or isomer. In one embodiment, the Prx1 comprises the amino acid sequence shown for NCBI Reference Sequence: NP_{—}859047.1 in the Aug. 23, 2009 entry which is incorporated herein by reference. In one embodiment, the binding of a Prx1 decamer to TLR4 is inhibited.

In our characterization of Prx1 as a TLR4 ligand, we show that incubation of Prx1 with thioglycollate (TG)-elicited murine macrophages or immature bone marrow derived dendritic cells resulted in Toll-like receptor 4 (TLR4) dependent secretion of TNF-α and IL-6 and dendritic cell maturation. Optimal secretion of cytokines in response to Prx1 was dependent upon serum and required CD14 and MD2. Binding of Prx1 to TG-macrophages occurred within minutes and resulted in TLR4 endocytosis. Prx1 interaction with TLR4 was independent of its peroxidase activity and appeared to be dependent upon its chaperone activity and ability to form decamers. Cytokine expression occurred via the TLR-MyD88 signaling pathway, which resulted in nuclear translocation and activation of NFκB. These and other data as described more fully herein show that extracellular Prx1 binds to TLR4 and induces biochemical cascades known to be affected by TLR4-ligand binding.

While Prx1 is known to be elevated in tumors, the role of elevated Prx1 in the tumors is unclear. However, we demonstrate that reduction of Prx1 levels by expression of shRNA specific for Prx1 results in inhibition of prostate tumor growth in two murine tumor models of prostate cancer (CaP). Interestingly, the loss of Prx1 had no effect on tumor cell growth in vitro or cell survival in vivo. In connection with this, examination of the tumors revealed that Prx1 expression correlated with the presence of tumor vessels; in the absence of Prx1, the number of vessels was significantly reduced and less mature. Furthermore, the vessels that were present in tumors with reduced Prx1 levels were less functional than vessels that were not associated with cells that have reduced Prx1 levels.

As is known in the art, angiogenesis is regulated by a number of growth factors, including vascular endothelial growth factor (VEGF). We demonstrate that inhibition of Prx1 expression leads to a loss of VEGF expression within the tumor microenvironment. Therefore, in one embodiment, the invention provides a method for reducing VEGF mRNA, VEGF protein, or a combination thereof in the tumor. The method comprises administering to an individual who has a tumor a compostion comprising an agent capable of inhibiting binding of Prx1 to TLR4.

The function of extracellular/secreted Prx1 is unknown. However, we demonstrate that secreted Prx1 binds to toll-like receptor 4 (TLR4) and stimulates the release of VEGF. Furthermore, we show that Prx1 stimulates VEGF promoter activity and this stimulation is dependent upon TLR4 signaling. We further demonstrate that Prx1 stimulates expression of VEGF mRNA and protein, that Prx1 stimulation of VEGF mRNA is regulated by the transcription factor HIF-1α. We also show that this is dependent upon Prx1 interaction with TLR4, and that Prx1 stimulation of HIF-1α activity is dependent upon NF-κB activation of HIF-1α.

Angiogenesis and formation of new vessels is due in part to proliferation and migration of endothelial cells. We demonstrate that Prx1 stimulates migration of endothelial cells in vivo and in vitro and the stimulation of migration is dependent upon TLR4. We also show that Prx1 also stimulates proliferation of endothelial cells in a TLR4 dependent manner. Further, we demonstrate that the ability of Prx1 to bind to TLR4 is dependent upon it chaperone activity, and that Prx1 mutants that lack chaperone activity can not stimulate endothelial cell proliferation. Further still, tumor cells that express Prx1 are unable to grow in mice that lack TLR4. Thus, it is expected that inhibition of Prx1 or Prx1 chaperone activity will prevent activation of TLR4, block tumor angiogenesis and result in inhibition and/or prevention of tumor growth.

It is expected that the invention will be suitable for inhibiting angiogenesis in any type of tumor. In one embodiment, the individual has a prostate tumor. In another embodiment, the individual has a tumor selected from thyroid, lung, bladder, breast, and oral cancer tumors.

In various embodiments of the invention, inhibition of Prx1 can be achieved by using any method and/or agent that inhibits Prx1 chaperoning and/or Prx1 binding to TLR4. It is preferable to interrupt Prx1 binding to TLR4 by inhibiting extracellular (secreted) Prxd1 from binding to TLR4, without interfering with Prx1 synthesis and its intracellular activity.

Inhibition of extracellular Prx1 binding to TLR4 according to the invention can be achieved using any method or agent, such as, for example, antibodies specific for Prx1, small drug compounds, including but not necessarily compounds that presently exist in chemical libraries and which can be identified as being capable of inhibiting Prx1 binding to and/or activation of TLR4. In an alternative embodiment, Prx1 binding to TLR4 can be achieved by reducing the intracellular synthesis of Prx1, which results in a reduction of secreted (extracellular) Prx1. For example, RNAi mediated degradation of Prx1 mRNA by, for example, using a shRNA specific for Prx1 can be used.

In various alternative embodiments, the agent that inhibits binding of Prx1 to TLR4 is an agent that inhibits Prx1 multimer formation. For example, it is expected that inhibition of Prx1 decamers will inhibit Prx1 binding to TLR4. Accordingly, any composition that can inhibit Prx1 multimerization can be used in the method of the invention. In one embodiment, the agent that inhibits Prx1 mulimerization is a fragment of Prx1, such as a Prx1 peptide or polypeptide, or an antibody to Prx1, that binds to Prx1 at one or more multimerization sites and therefore sterically precludes formation of Prx1 decamers.

In one embodiment, the agent used to inhibit binding of Prx1 to TLR4 is an antibody that binds to Prx1. The antibodies used in the invention will accordingly bind to Prx1 such that the binding of the antibody interferes with the activity of the TLR4 receptor and/or interferes with Prx1 binding to TLR4. The antibody may sterically hinder TLR4 binding, or it may inhibit Prx1 multimerization.

Antibodies that recognize Prx1 for use in the invention can be polyclonal or monoclonal. It is preferable that the antibodies are monoclonal. Methods for making polyclonal and monoclonal antibodies are well known in the art.

It is expected that antigen-binding fragments of antibodies may be used in the method of the invention. Examples of suitable antibody fragments include Fab, Fab′, F(ab′)₂, and Fv fragments. Various techniques have been developed for the production of antibody fragments and are well known in the art.

It is also expected that the antibodies or antigen binding fragments thereof may be humanized. Methods for humanizing non-human antibodies are also well known in the art (see, for example, Jones et al., Nature, 321:522-525 (1986); Riechmann et al., Nature, 332:323-327 (1988); Verhoeyen et al., Science, 239:1534-1536 (1988)).

Compositions comprising an agent that can inhibit Prx1 binding to TLR4 for use in therapeutic purposes may be prepared by mixing the agent with any suitable pharmaceutically acceptable carriers, excipients and/or stabilizers. Some examples of compositions suitable for mixing with the agent can be found in: Remington: The Science and Practice of Pharmacy (2005) 21st Edition, Philadelphia, Pa. Lippincott Williams & Wilkins.

Those skilled in the art will recognize how to formulate dosing regimes for the agents of the invention, taking into account such factors as the molecular makeup of the agent, the size and age of the individual to be treated, and the type and stage of disease.

Compositions comprising an agent that inhibits Prx1 binding to TLR4 can be administered to an individual using any available method and route suitable for drug delivery, including parenteral, subcutaneous, intraperitoneal, intrapulmonary, and intranasal. Parenteral infusions include intramuscular, intravenous, intraarterial, intraperitoneal, and subcutaneous administration.

Administration of the agent can be performed in conjunction with conventional therapies that are intended to treat a disease or disorder associated with the antigen. Such treatment modalities include but are not limited to chemotherapies, surgical interventions, and radiation therapy.

The amino acid sequence of Prx1 is known in the art. The secreted form of Prx1, the binding of which to TLR4 is inhibited by practicing the method of the invention, can be any form of Prx1 expressed by any individual. In one embodiment, the Prx1 has the decamer structure described in the literature.

The following examples are intended to illustrate but not limit the invention.

### Example 1

This Example provides a description of the materials and methods used in performance of embodiments of the invention.

### Materials

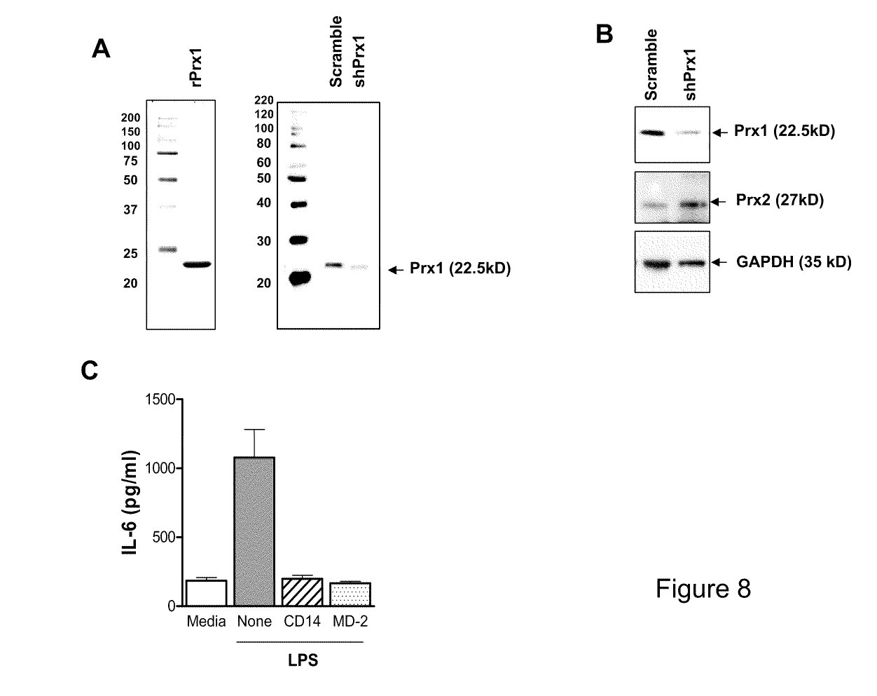

Lipopolysaccharide (LPS, Escherichia coli serotype 026:B6) polymyxin B sulfate salt, bovine serum albumin (BSA), and ovalbumin (OVA) were obtained from Sigma-Aldrich (St. Louis, Mo.). 7-Amino-Actinomycin D (7-AAD) and thioglycollate brewer modified media was purchased from (Becton Dickinson, La Jolla, Calif.). Capture and detection antibodies for IL-6 and TNF-α used in Luminex assays, as well as protein standards, were purchased from Invitrogen (Carlsbad, Calif.). Antibodies specific for CD11b, Gr-1, F4/80, and all isotypes were purchased from PharMingen (Mountain View, Calif.). Antibodies against TLR2, TLR4, and NFκB subunits were purchased from Santa Cruz Biotechnology (Santa Cruz, Calif.). Blocking antibodies against MD2 and CD14 were purchased from Santa Cruz Biotechnology. The phycoerythrin (PE) conjugated anti-TLR4 antibody was purchased from eBioscience (San Diego, Calif.). Antibodies specific for Prx1 were obtained from Lab Frontier (Seoul, South Korea); this antibody is specific for Prx1 and detects only a single band in Western analysis of cells that express Prx1 (FIG. 8A).

### Animals and Cell Lines

C57BL/6NCr (TLR4^{+/+} and TLR2^{+/+}), C57BL/10ScNJ (TLR4^{-/-}), B6.129-Tlr2^{tm1Kir/J} (TLR2^{-/-}), C3H/HeNCr (TLR4^{+/+}), and C3H/HeNJ (TLR4^{-/-}) pathogen-free mice were purchased from The Jackson Laboratory (Bar Harbor, Me.). Animals were housed in microisolator cages in laminar flow units under ambient light. The mice were maintained in a pathogen-free facility at Roswell Park Cancer Institute (Buffalo, N.Y.). The Institutional Animal Care and Use Committee approved both animal care and experiments.

The role of Prx1 in vivo was determined by injecting either C57BL/6NCr or C57BL/10ScNJ mice intravenously with 90 ug Prx1 (˜1000 nM). Cardiac punctures were performed 2 hours later. Serum was obtained by incubation of blood at 4° C. overnight then samples were centrifuged and supernatants collected.

The cultured mouse macrophage cell line (RAW264.7) was maintained in Dulbeco's Modified Eagle Media (DMEM) containing 10% defined fetal bovine serum and 100 U/ml penicillin and 100 ug/ml streptomycin at 37° C. and 5.0% CO₂. RAW264.7 cells were transfected with the pcDNA3.1 plasmid containing either control or MyD88 dominant negative (DN) encoding oligonucleotides using FuGENE 6 (Invitrogen, Carlsbad, Calif.) according to the manufacturer's protocol. The transfected cells were then selected using G418 for cells expressing the control or MyD88 DN. Cells were then stimulated with buffer, Prx1, or LPS for 24 h and culture media was harvested for IL-6 cytokine analysis by ELISA.

The retroviral short hairpin RNA expression constructs and retroviral infection procedure used to create a knock down of Prx1 in the lung cancer cell line (A549) is known in the art (Kim, et al; (2007) Cancer Res. 67:546-554, Park, et al. Cancer Res. (2007) 67:9294-9303; Park, et al. 2006. Cancer Res. 66:5121-5129, the disclosures of which are incorporated herein by reference).

### Macrophage and Dendritic Cell Isolation

Peritoneal elicited macrophage cells from mice were obtained by an intraperitoneal injection of 1.0 ml of 3.0% (w/v) thioglycollate media (TG). Four days after injection, mice were sacrificed and macrophages were obtained by peritoneal lavage. Macrophages were enriched by adherence selection for 1 h in complete media (DMEM supplemented with 10% defined FBS, 100 U/ml penicillin and 100 μg/ml streptomycin) and were characterized through FACS analysis for expression of CD11b, Gr1 and F4/80 using standard techniques; cells that were CD11b⁺Gr1⁻F4/80⁺ were identified as macrophages.

Immature bone marrow derived dendritic cells were generated by culture of bone marrow derived cells in GM-CSF using standard techniques. Dendritic cells were identified by the expression of CD11c.

### Protein Purification

Recombinant human Prx1, Prx1C52S, and Prx1C83S proteins were purified as described previously (Kim, et al. 2006. Cancer Res. 66:7136-7142; Lee, et al. 2007. J. Biol. Chem. 282:22011-22022, the disclosures of each of which are incorporated herein by reference). Briefly, bacterial cell extracts containing recombinant proteins were loaded onto DEAE-sepharose (GE Healthcare, USA) and equilibrated with 20 mM Tris-Cl (pH 7.5). The proteins were dialyzed with 50 mM sodium phosphate buffer (pH 6.5) containing 0.1 M NaCl. The unbound proteins from the DEAE column containing Prx1, Prx1C52S, or Prx1C83S were pooled and loaded onto a Superdex 200 (16/60, GE Healthcare, USA), and equilibrated with 50 mM sodium phosphate buffer (pH 7.0) containing 0.1 M NaCl. The fractions containing Prx1, Prx1 C52S, or Prx1C83S were pooled and stored at −80° C. Endotoxin levels of purified proteins were quantified with a Limulus Amebocyte Lysate Assay (Lonza, Walkersville, Md.) according to manufacturer's directions. Prx1, Prx1C52S, and Prx1C83S were found to contain 14.14±0.050 EU/ml, 14.07±0.67 EU/ml, and 14.17±0.025 EU/ml respectively.

### Cytokine Analysis.

Adherent TG-elicited macrophage cells were washed 5-10 times with PBS, to remove any non-adherent cells. Once washed, complete media containing purified Prx1, Prx1C52S, Prx1C83S, or LPS at the specified concentrations were added in the presence or absence of Prx1, MD-2 and CD14 blocking or control antibodies. In the indicated experiments Prx1 proteins or LPS were incubated with polymyxin B or were boiled for 20 minutes prior to addition. After 24 h the supernatant was collected and analyzed by cytokine specific ELISA or the Luminex multiplex assay system. Serum samples were collected as indicated above and IL-6 levels were determined by ELISA. TNF-α and IL-6 ELISA kits were purchased from BD Bioscience (Franklin Lakes, N.J.) and assays were completed according to manufacturer's instructions.

Luminex analyses were performed by the Institute Flow Cytometry Facility in 96-well microtiter plates (Multiscreen HV plates, Millipore, Billerica, Mass.) with PVDF membranes using a Tecan Genesis liquid handling robot (Research Triangle Park, N.C.) for all dilutions, reagent additions and manipulations of the microtiter plate. Bead sets, coated with capture antibody were diluted in assay diluents, pooled and approximately 1000 beads from each set were added per well. Recombinant protein standards were titrated from 9,000 to 1.4 pg/ml using 3-fold dilutions in diluent. Samples and standards were added to wells containing beads. The plates were incubated at ambient temperature for 120 min on a rocker, and then washed twice with diluent using a vacuum manifold to aspirate. Biotinylated detection antibodies to each cytokine were next added and the plates were incubated 60 min and washed as before. Finally, PE conjugated streptavidin was added to each well and the plates were incubated 30 min and washed. The beads were resuspended in 100 μl wash buffer and analyzed on a Luminex 100 (Luminex Corp., Austin, Tex.). Each sample was measured in duplicate, and blank values were subtracted from all readings. Using BeadView Software (Millipore) a log regression curve was calculated using the bead MFI values versus concentration of recombinant protein standard. Points deviating from the best-fit line, i.e. below detection limits or above saturation, were excluded from the curve. Sample cytokine concentrations were calculated from their bead's mean fluorescent intensities by interpolating the resulting best-fit line. Samples with values above detection limits were diluted and reanalyzed.

### FITC Labeling of Proteins

BSA, Prx1, Prx1C52S, and Prx1C83S proteins were conjugated to FITC using a FITC conjugation kit (Sigma, St. Louis, Mo.). A twenty-fold excess of FITC and individual proteins were dissolved into a 0.1M sodium bicarbonate/carbonate buffer (pH adjusted to 9.0); the mix was incubated for 2 h at room temperature with gentle rocking. The excess free FITC was removed with a Sephadex G-25 column (Pharmacia, Piscataway, N.J.). Proteins amounts were quantified using a standard Lowry assay. The F:P (fluorescence:protein) ratio was calculated according to the manufacturer's instructions using the optical density at 495 nm (FITC absorbance) and 280 nm (protein absorbance). FITC per nM protein for BSA, Prx1, Prx1 C52S, and Prx1 C83S were 31.00±1.92, 38.52±2.39, 74.49±2.64, and 44.44±2.64 respectively.

### Saturation Assay

FITC-conjugated BSA, Prx1, Prx1C52S, and Prx1C83S were diluted in 1.0% BSA in PBS to the specified concentrations and a total reaction volume of 100 μL. These mixtures were incubated with 1.0×10⁶ cells/mL for 20 min on ice to prevent internalization. Cells were washed twice with 1% BSA in PBS and cells were incubated to demonstrate viable from nonviable cells with 7-AAD, less than 30 min before FACsCalibur analysis. Data was acquired from a minimum of 20,000 cells, stored in collateral list mode, and analyzed using the WinList processing program (Verity Software House, Inc., Topsham, Me.). Cells positive for 7-AAD (nonviable) were gated out of the events. FITC-conjugated BSA was used as a negative binding control and for mutant studies variations in FITC labeling were normalized by FITC labeling per nM proteins.

### Competition Assay

Unlabeled OVA, Prx1, Prx1C52S, and Prx1C83S were briefly mixed with FITC conjugated Prx1 at the specified concentrations in 100 μL 1.0% BSA in PBS. The mixture was incubated for 20 min on ice, before washing twice with 1.0% BSA in PBS. Cells were then incubated with 7-AAD and analyzed within 30 min by flow cytometry. OVA was used as a negative competition control in all competition assays. Data was acquired from a minimum of 20,000 cells, stored in collateral list mode, and analyzed using the WinList processing program (Verity Software House, Inc., Topsham, Me.). When using WinList to analyze results, 7-AAD positive cells were gated out of the events.

### Immunoprecipitation

Immunoprecipitation was carried out with 500 μg of cell lysates and 4 μg of anti-TLR4 or anti-TLR2 overnight at 4° C. After the addition of 25 μL of Protein G-agarose (Santa Cruz Biotechnology), the lysates were incubated for an additional 4 h. To validate specific protein interactions, goat IgG (Santa Cruz Biotechnology) or mouse IgG (Santa Cruz Biotechnology) was used as negative control. The beads were washed thrice with the lysis buffer, separated by SDS-PAGE, and immunoblotted with antibodies specific for Prx1. The proteins were detected with the ECL system (Biorad).

### Co-Localization of Prx1/TLR4 and NFκB Translocation

Colocalization experiments were performed by the addition of 200 nM FITC-labeled Prx1 and PE-conjugated anti-TLR4 to the media of TG-elicited macrophages and kept at 37° C. for the indicated times before being transferred to ice, fixed and analyzed. Immunostaining to detect the nuclear translocation of NFκB was performed in the following manner. TG-elicited macrophages obtained from C3H/HeNCr (TLR4^{+/+}) and C3H/HeNJ (TLR4^{-/-}) were treated with 200 nM Prx1. After the indicated times at 37° C. the cells were then scraped and collected in tubes, washed twice in wash buffer (2% FBS in phosphate-buffered saline), and then fixed in fixation buffer (4% paraformaldehyde in phosphate-buffered saline) for 10 min at room temperature. After washing, the cells were re-suspended in Perm Wash buffer (0.1% Triton X-100, 3% FBS, 0.1% sodium azide in phosphate-buffered saline) containing 10 μg/ml anti-NF B p65 antibody (Santa Cruz Biotechnology) for 20 min at room temperature. The cells were then washed with Perm Wash buffer and resuspended in Perm Wash buffer containing 7.5 μg/ml FITC conjugated F(ab′)₂ donkey anti-rabbit IgG for 15 min at room temperature. Cells were washed twice in Perm Wash buffer and re-suspended in 1% paraformaldehyde containing 5 μM DRAQ5 nuclear stain (BioStatus) for 5 min at room temperature.

### Image Analysis

Co-localization of Prx1 and TLR4 and nuclear translocation of NFκB were analyzed with the ImageStream® multispectral imaging flow cytometer (Amnis Corp., Seattle, Wash.). At least 5000 events were thus acquired for each experimental condition and the corresponding images were analyzed using the IDEAS® software package. A hierarchical gating strategy was employed using image-based features of object contrast (gradient RMS) and area versus aspect ratio to select for in-focus, single cells. Co-localization and nuclear translocation was determined in each individual cell using the IDEAS® similarity feature which is a log transformed Pearson's correlation coefficient of the intensities of the spatially correlated pixels within the whole cell, of the Prx1 and TLR4 images or NFκB and DRAQ5 images, respectively The similarity score is a measure of the degree to which two images are linearly correlated.

### Electrophoretic Mobility Shift Assay (EMSA)

EMSA was performed using conventional techniques. Briefly, 10 μg of nuclear protein was incubated with γ-³²P-labeled double-stranded NFκB oligonucleotide in 20 μL of binding solution containing 10 mM HEPES (pH 7.9), 80 mM NaCl, 10% glycerol, 1 mM DTT, 1 mM EDTA, 100 μg/mL poly(deoxyinosinic-deoxycytidylic acid). The DNA-protein complexes were resolved on a 6% polyacrylamide gel under non-denaturing conditions at 200 V for 2 h at 4° C. Gels were dried and then subjected to autoradiography.

### Statistical Analysis

Statistical analyses were performed using a standardized t-test with Welch's correction, where equal variances were not assumed, to compare experimental groups. Differences were considered significant when P values were ≦0.05.

### Example 2

This Example provides a description of results obtained using the materials and methods described in Example 1.

Prx1 Stimulation of Cytokine Secretion from DCS and TG-Macrophages and Maturation of DCs is Dependent Upon TLR4

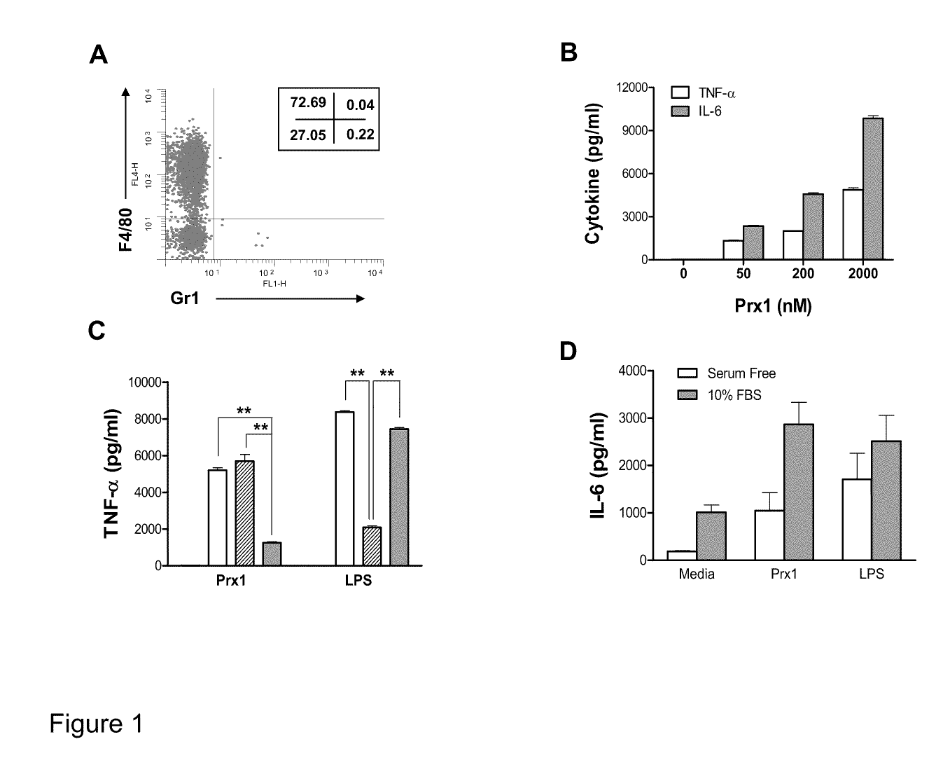

Thioglycolate (TG)-elicited murine macrophages were used to assess the ability of Prx1 to stimulate cytokine secretion. Macrophage phenotype was assessed by analysis of peritoneal exudate cell populations for CD11b, Gr1, and F4/80 expression. The isolated populations were greater than 99% CD11b⁺ and of the CD11b⁺ cell population a majority were Gr1⁻, F4/80⁺ (FIG. 1A). Stimulation of TG-elicited macrophages with Prx1 resulted in the dose dependent secretion of TNF-α and IL-6 that was significantly greater than that observed in unstimulated cells at all doses (P≦0.01; FIG. 1B). Pre-incubation of Prx1 with the endotoxin inactivator polymixin B had no significant effect on Prx1 stimulation of cytokine secretion (FIG. 1C); in contrast, denaturing of Prx1 significantly reduced its ability to stimulate cytokine secretion (P<0.01).

Stimulation of cytokine secretion by TG-elicited macrophages following incubation with Prx1 was significantly diminished in the absence of serum (P≦0.01; FIG. 1D); however even in serum free conditions, incubation of TG-elicited macrophages with Prx1 significantly increased IL-6 secretion (P≦0.005 when compared to secretion by cells incubated in serum free media). Prx1 was also able to stimulate cytokine secretion from the cultured dendritic cell line, DC1.2, and the murine macrophage cell line, RAW264.7 (data not shown).

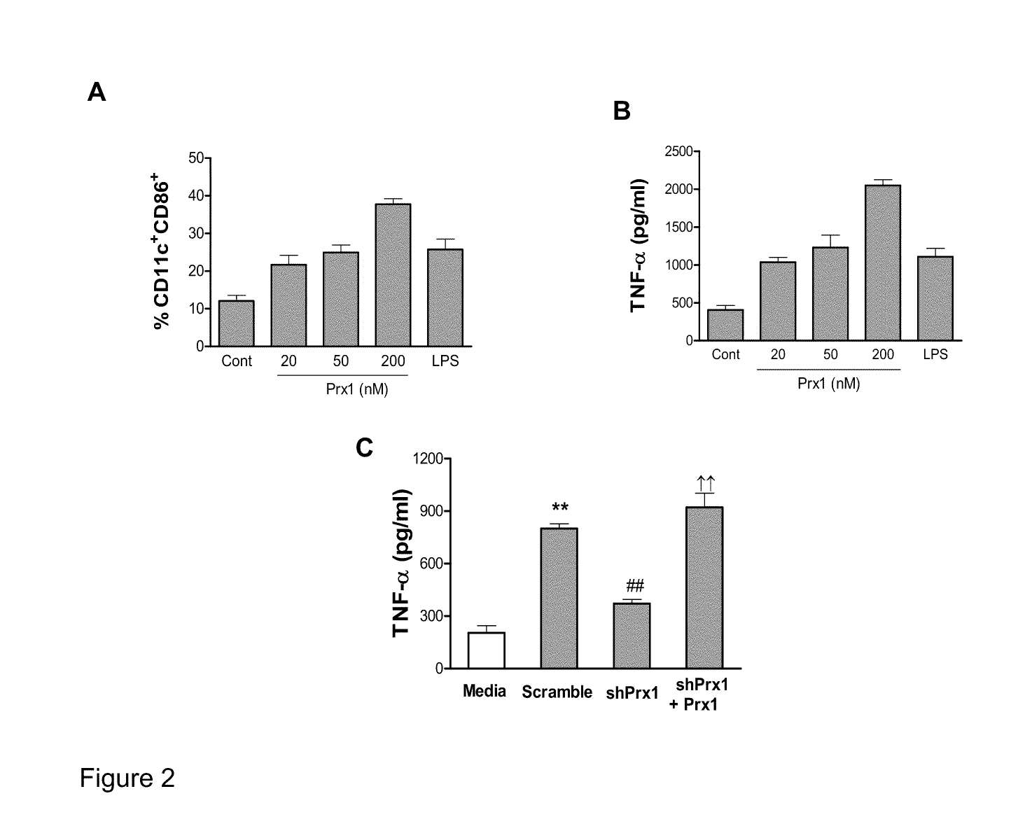

Exogenous Prx1 was able to induce maturation and activation of immature bone marrow derived DCs (iBMDCs). iBMDCs were incubated with increasing concentrations of Prx1 for 24 h and examined for cell surface expression of co-stimulatory molecules and secretion of TNF-α. Addition of Prx1 led to significant dose dependent increase in cell surface expression of the co-stimulatory molecule, CD86 (FIG. 2A) and TNF-α secretion (FIG. 2B) at all doses tested (P≦0.01 when compared to control).

It is possible that enhanced secretion of cytokines from iBMDCs and TG-elicited macrophages upon addition of exogenous recombinant Prx1 is a phenomena of the recombinant protein and not physiologically relevant. To begin to determine whether Prx1 could promote cytokine secretion in a physiologic context, TG-elicited macrophages were incubated for 24 h in the presence of supernatant collected from Prx1-secreting tumor cells or supernatant collected from tumor cells engineered to express shRNA specific for Prx1. Expression of shRNA resulted in reduced expression of Prx1, but not Prx2 FIG. 8B). Incubation of TG-elicited macrophages with supernatants of tumor cells engineered to express a non-specific shRNA, resulted in enhanced expression of TNF-α (Sc, FIG. 2C; P≦0.0001 when compared to media). In contrast, TG-elicited macrophages incubated with supernatants collected from tumor cells expressing reduced levels of Prx1 secreted significantly lower levels of TNF-α (P≦0.0001 when compared to incubation with supernatant harvested from cells expressing control shRNA; FIG. 2C); addition of exogenous Prx1 to these supernatants restored TNF-α secretion from TG-elicited macrophages (shPrx1+Prx1; P≦0.003 when compared to incubation with supernatant harvested from cells expressing shRNA specific for Prx1).

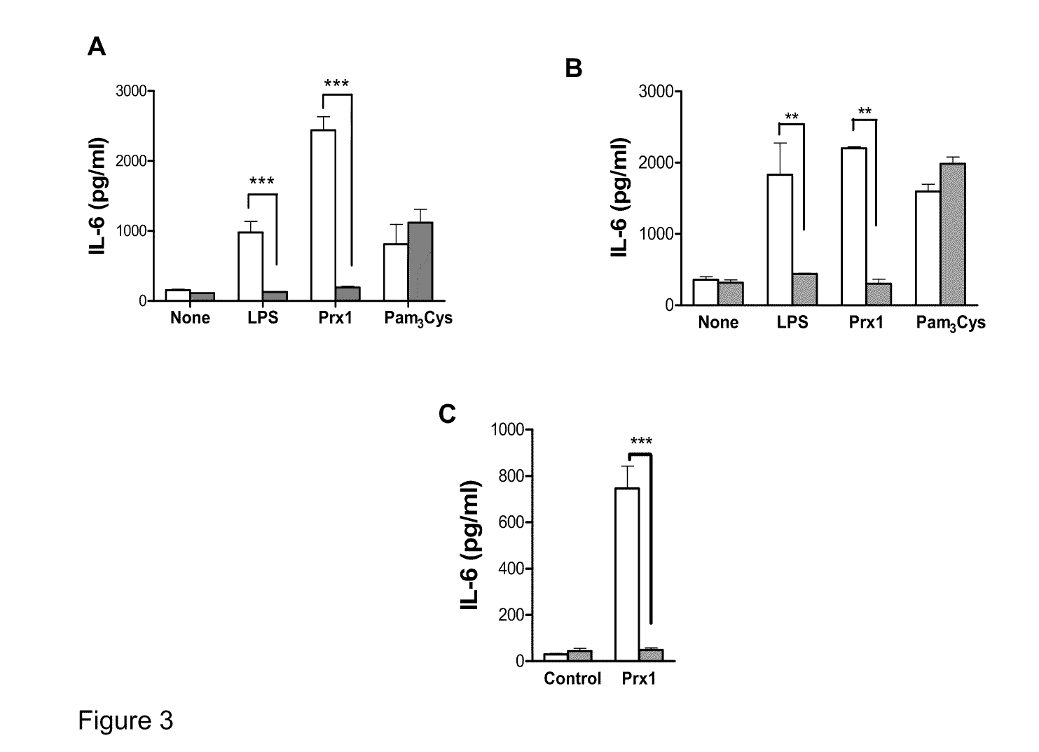

To test whether Prx1 activation of iBMDCs and TG-elicited macrophages was dependent upon TLR4, iBMDCs and TG-elicited macrophages were isolated from C57BL/6NCr (TLR4^{+/+}) and C57BL/10ScNJ (TLR4^{-/-}) mice and stimulated with Prx1, LPS or Pam₃Cys, a TLR2 agonist. The results indicate that Prx1, LPS, and Pam₃Cys stimulate cytokine secretion from iBMDCs (FIG. 3A) and macrophages isolated from C57BL/6NCr mice (FIG. 3B); only Pam₃Cys stimulated cytokine secretion from iBMDCs and macrophages isolated from C57BL/10ScNJ mice (P≦0.01 when compared to cytokine secretion by cells isolated form C57BL/NCr mice).

The ability of Prx1 to induce TLR4 dependent inflammation in vivo was tested by i.p. injection of recombinant Prx1 into either C57BL/6NCr (TLR4^{+/+}) or C57BL/10ScNJ (TLR4^{-/-}) mice. Blood was collected 2 h post injection and the extent of systemic inflammation was determined by assessing the level of systemic IL-6 (FIG. 3C). Injection of Prx1 resulted in a significant increase in systemic IL-6 levels (P≦0.0002) in C57BL/6NCr (TLR4^{+/+}) mice, but had no significant effect on systemic IL-6 levels in C57BL/10ScNJ (TLR4^{-/-}) mice.

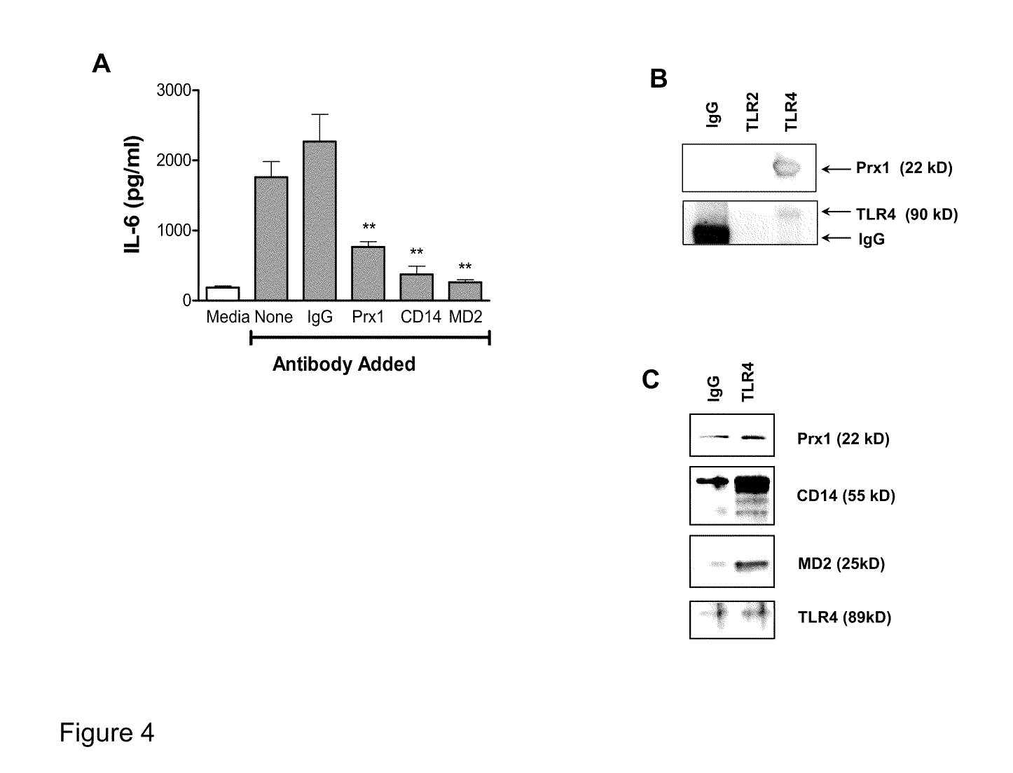

The reduced expression of cytokines by TG-elicited macrophages following incubation with Prx1 in the absence of serum (FIG. 1D) suggests that serum proteins may contribute to optimal Prx1/TLR4 interaction. Many TLR4 ligands interact with TLR4 as part of a larger complex that can include CD14 and/or MD2. To determine whether Prx1 enhancement of cytokine secretion from TG-elicited macrophages involves CD14 or MD2, cells were incubated with Prx1 or LPS in the presence of blocking antibodies to MD2, CD14 or control IgG (FIG. 4A). Addition of blocking antibodies to Prx1, CD14 or MD2 significantly inhibited the ability of Prx1 to stimulate IL-6 secretion from TG-elicited macrophages when compared to that induced by Prx1 in the presence of control IgG (P≦0.01). Blocking antibodies to CD14 and MD2 also blocked cytokine secretion in LPS stimulated cells (FIG. 8C).

To further demonstrate the interaction Prx1 and TLR4/MD2/CD14, TG-elicited macrophage cell lysates were incubated with isotype control antibodies or antibodies specific for TLR4 or TLR2 (FIG. 4B). The antibody complexes were isolated and immunoblotting was performed using antibodies to Prx1; Prx1 was only found in the lysates immunoprecipitated with TLR4 (FIG. 4B). The TLR4/Prx1 complexes isolated from Prx1 treated cells also contained CD14 and MD2 (FIG. 4C), confirming the finding that Prx1 interacts with TLR4 in a complex that contains both CD14 and MD2.

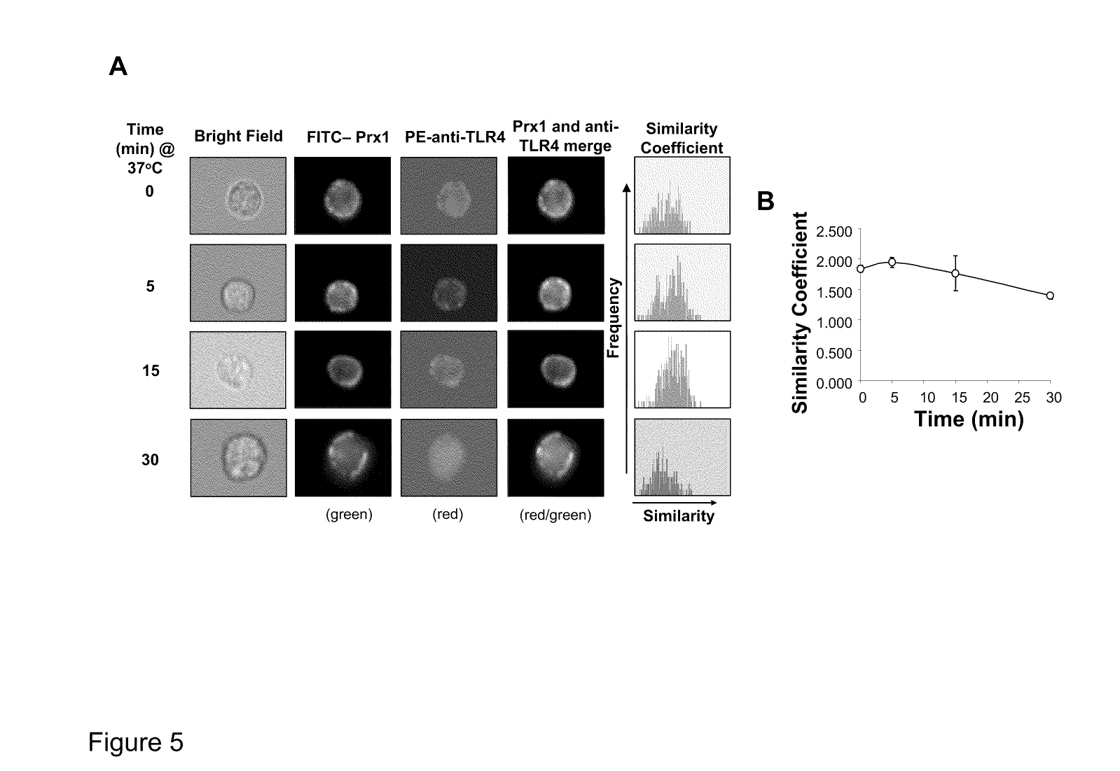

The kinetics of the Prx1 and TLR4 interaction was determined using image stream analysis (Amnis) to examine co-localization of the two molecules. TG-elicited macrophages were incubated with FITC-labeled Prx1 and PE-conjugated anti-TLR4 antibodies. The merged images of representative cells indicate that Prx1 and TLR4 localize together on the membrane of the macrophage within 5 minutes and that by 30 min, TLR4 and a portion of the Prx1 molecules have been internalized (FIG. 5A). The histograms to the right of the merged images are a statistical analysis of the similarity of FITC-Prx1 and PE-anti-TLR4 in 5,000 cells on a pixel-by-pixel basis. A shift of this distribution to the right indicates a greater degree of similarity. The average similarity coefficient at each time point was demonstrated in FIG. 5B. At all time points there was a high similarity of Prx1 and TLR4 staining (similarity coefficients >1), indicating a co-localization Prx1 and TLR4. These results confirm that Prx1 and TLR4 interact on the cell surface and that at least of portion of the Prx1 is internalized with TLR4.

### Stimulation of Cytokine Secretion and Binding to TLR4 Depends Upon Prx1 Structure

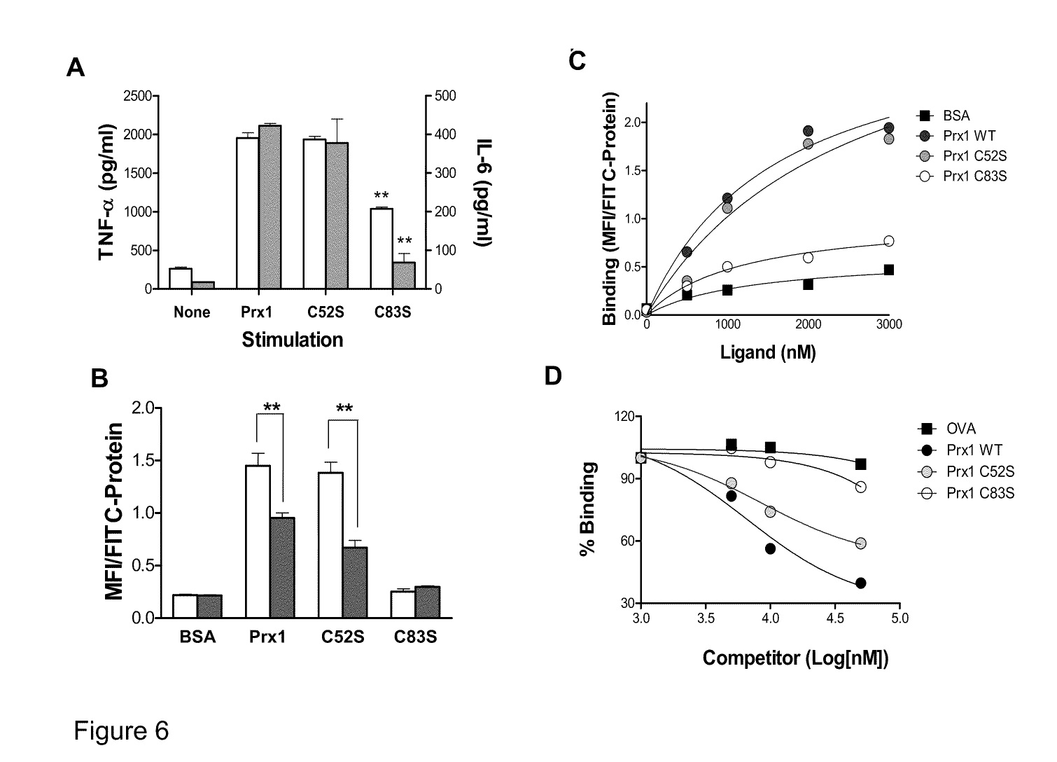

Prx1 acts as both a peroxidase and a protein chaperone (Wood, et al. (2003) Trends Biochem. Sci. 28:32-40). To determine whether the ability of Prx1 to stimulate cytokine secretion from TG-elicited macrophages was related to its peroxidase activity and/or chaperone activity, two Prx1 mutants were examined. The Prx1C52S mutant lacks peroxidase activity but retains the decamer structure needed for chaperone activity; Prx1C83S exists mainly as a dimer, has reduced chaperone activity and intact peroxidase activity. Cytokine secretion following Prx1C52S stimulation of TG-elicited macrophages was not significantly distinct from that observed following stimulation with Prx1 (FIG. 6A); however, TG-elicited macrophages stimulated with Prx1C83S displayed a significant reduction in cytokine secretion (P≦0.01).

Prx1 binding to TG-elicited macrophages was dependent upon the presence of TLR4 as binding of Prx1 and the enzymatic null mutant (Prx1C52S) was significantly decreased in the absence of TLR4 (FIG. 6B). Prx1C83S binding was minimal to either TLR4 expressing or non-expressing macrophages, confirming that Prx1 interaction with TLR4 is peroxidase independent and structure dependent.

Saturation binding (FIG. 6C) and competition analyses (FIG. 6D) were used to determine the K_{d}, and Kᵢ values for Prx1 binding to the surface of TG-elicited macrophages. The K_{d} for Prx1 binding to TG-elicited macrophages was 1.6 mM and the Kᵢ was 4.1 mM (Table 1).

### Prx1 Stimulation of Cytokine Secretion is MyD88-Dependent and Leads to TLR4-Dependent Translocation of NFκB to the Nucleus

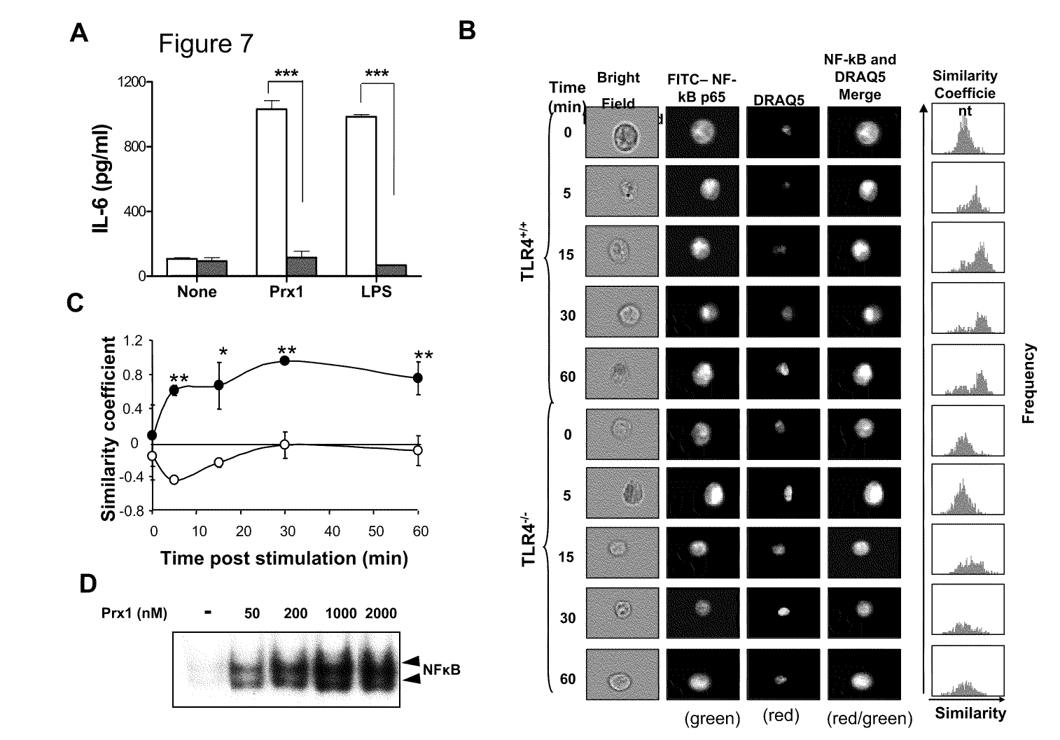

The consequential downstream signaling events of ligand-mediated activation of TLR4 can be MyD88 dependent or independent. Prx1 was used to stimulate cytokine expression from RAW264.7 cells expressing dominant negative (DN) MyD88 protein. IL-6 secretion following Prx1 stimulation is dependent on MyD88 function (FIG. 7A), indicating that Prx1 activates the MyD88 signaling cascade, which can lead to activation of NFκB.

To determine if Prx1/TLR4 interaction leads to NFκB activation, NFκB translocation following Prx1 stimulation was analyzed in macrophages isolated from C3H/HeNCr and C3H/HeNJ mice. C3H/HeNJ mice have a mutation in the TLR4 ligand binding domain that prevents ligand binding. TG-elicited macrophages from C3H/HeNCr and C3H/HeNJ mice were incubated with 200 nM Prx1 at 37° C. for the indicated times, transferred to ice and incubated with antibodies against NFκB p65; the nuclear stain DRAQ5 was added 15 minutes prior to image stream analysis. Prx1 incubation with macrophages isolated from C3H/HeNCr mice triggered NFκB translocation within 5 min and nuclear localization was apparent for up to 60 min (FIG. 7B). In contrast Prx1 incubation with macrophages isolated from C3H/HeNJ mice did not trigger NFκB translocation (FIG. 7B). The histogram to the right of the merged image column depicts the similarity of NFκB and the nuclear stain on a pixel-by-pixel basis. Prx1 stimulation led to NFκB translocation to the nucleus in a TLR4 dependent manner as demonstrated by the positive similarity coefficient observed following Prx1 stimulation of C3H/H3NCr TG-elicited macrophages, which was decreased following Prx1 stimulation of C3H/HeNJ TG-elicited macrophages (FIG. 7C). The ability of Prx1 to activate NF-κB was confirmed by EMSA, which indicated that incubation of macrophages with Prx1 resulted in a dose dependent increase in NFκB DNA binding activity (FIG. 7D).

It will be recognized by those skilled in the art that the foregoing results are compelling evidence that Prx1 stimulates TLR4-dependent secretion of TNF-α and IL-6 from TG-elicited macrophages and DCs. Cytokine secretion was the result of TLR4 stimulation of the MyD88-dependent signaling cascade and resulted in activation and translocation of NFκB. Prx1 is an intercellular protein that is secreted from tumor cells and activated T cells. The ability of Prx1 to interact with TLR4 and stimulate the release of pro-inflammatory cytokines suggests that it may also act as an endogenous damage-associated molecular pattern molecule (DAMP).

HSP72 and HMGB1, which have also been classified as endogenous DAMPs, have been shown to interact with TLR4. Saturation and competition studies indicate that Prx1 has a K_{d} of ˜1.3 mM and a Kᵢ of ˜4.1 mM; extrapolation of data presented by Binder et al. (Binder, et al. 2000. J. Immunol. 165:2582-2587) implies that HSP72 has a K_{d} of 2.1-4.4 mM and a Kᵢ of 10-21.8 mM, suggesting that Prx1 interaction with TLR4 is stronger than that of HSP72. Binding affinities are not available for HMGB1.

Identification of TLR4 as a receptor for a recombinant protein may be complicated by the potential of the presence of LPS within a recombinant protein preparation. To account for this possibility in the results presented here, two controls were included in all of the performed studies. In the first control, recombinant proteins were combined with polymixin B prior to their addition to immune cells. Polymixin B is a powerful inactivator of LPS; pre-incubation of recombinant Prx1 with polymixin B had no effect on the ability of Prx1 to stimulate cytokine expression (FIG. 1). However pre-incubation of LPS with the same concentration of polymixin B significantly inhibited its ability to stimulate cytokine release. As a second control, Prx1 and LPS were boiled prior to addition to immune cells; denaturing Prx1 significantly inhibited its ability to stimulate cytokine release, but boiling had no effect on the ability of LPS to stimulate cytokine release. Finally, all of the recombinant proteins used in this study were prepared in the same fashion and following purification all were found to have equivalent levels of endotoxin (˜14 EU/ml), yet Prx1C83S stimulated significantly lower cytokine secretion and did not appear to bind to TLR4 expressing cells. Thus it appears as though the results demonstrating that Prx1 interacts with TLR4 are not due to the presence of LPS contamination.

Prx1, HSP72 and HMGB1 not appear to have significant structural similarity nor do these molecules appear to share homology with LPS. Prx1, HSP72 and HMGB1 are molecular chaperones and the lack of structural homology between HSP72/HMGB1 and other TLR4 ligands has led some to speculate that the chaperone cargo rather than the chaperone is being recognized by TLR4. In support of this hypothesis, recent studies have shown that HMGB1 binding to TLR9 is a result of TLR9 recognition of HMGB1/DNA complexes. Extracellular Prx1 is present as a decamer, which is associated with Prx1 chaperone activity (Wood, et al. 2002. Biochemistry 41:5493-5504, the disclosure of which is incorporated herein by reference) and our studies indicate that Prx1 binding to TLR4 was dependent upon the ability to form decamers (FIGS. 3 and 4B). Thus it is possible that Prx1 binding of TLR4 is due to recognition of its cargo rather than of Prx1 itself. Nevertheless, agents that interfere with Prx1 binding to TLR4 according to the invention are expected to inhibit angiogenesis.

The Prx1C83S mutant, which lacks chaperone activity and exists primarily as a dimer (Wood, et al. 2002. Biochemistry 41:5493-5504) did not appear to bind to TLR4 (FIG. 4B); however the purified mutant protein was able to stimulate cytokine secretion from macrophages (FIG. 4A). Assays for biological function are traditionally more sensitive than binding assays and it is possible that the interaction of the dimeric form of Prx1 with TLR4 was below the level of detection in the binding assay employed in these studies. A small portion of Prx1C83S is present as a tetramer, which may also be able to interact with TLR4 at a level that is below detection, but that is sufficient to stimulate cytokine secretion.

Prx1 stimulation of cytokine secretion was dependent on TLR4 and MyD88 (FIGS. 3, 4 and 5); however, FITC-labeled Prx1 did bind to macrophages isolated from TLR4^{-/-} (B10ScNJ) mice (FIG. 4B), albeit at a lower level than bound to macrophages isolated from TLR4^{+/+} (B6) mice. Examination of the interaction of Prx1 with TLR4 at a cellular level indicated that while a majority of the TLR4 was internalized upon Prx1 binding, at least a portion of the Prx1 remained on the cell surface (FIG. 3B/C). These findings could be the result of excess Prx1 or alternatively that Prx1 is binding to additional receptors. Other TLR4 binding DAMPs have been shown to bind to multiple danger receptors and in some cases DAMP binding to TLR4 requires co-receptors. PbA, the malaria homolog of Prx1 requires MD2 to bind to TLR4; our studies indicate that Prx1 stimulation of cytokine secretion is optimal in the presence of serum and that antibodies to CD14 and MD2 block cytokine secretion from Prx1 stimulated cells. Furthermore, immunoprecipated complexes of TLR4 and Prx1 contain MD2 and CD14, suggesting that these proteins contribute to the binding of Prx1 to TLR4. Moreover, as the following Example demonstrates, blocking Prx1 from binding to TLR4 can inhibit tumor angiognesis.

### Example 3

This Example provides a description of an embodiment of the invention wherein angiogenesis is a tumor is inhibited and further characterizes the effects of Prx1 on VEGF expression.

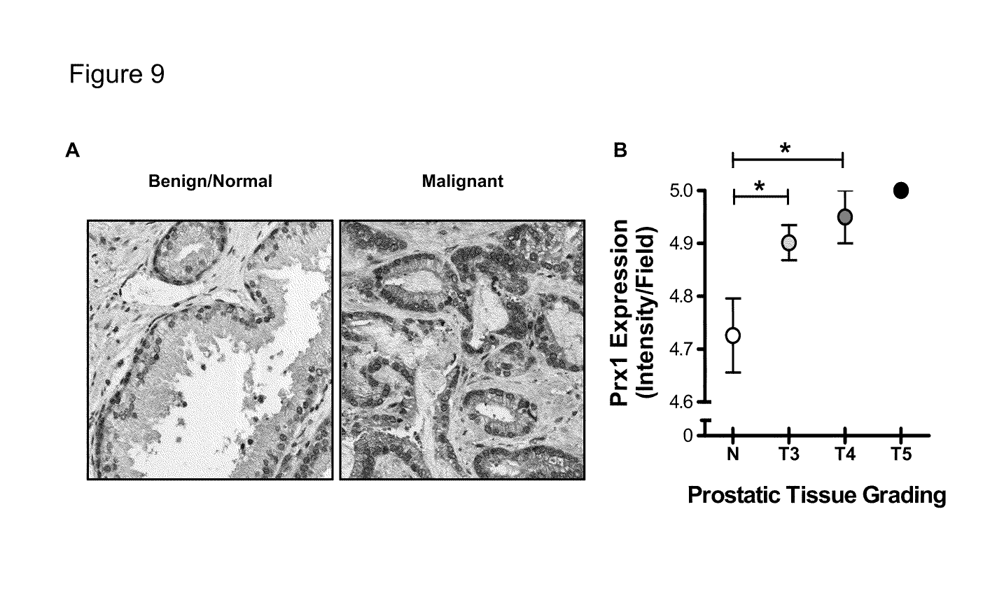

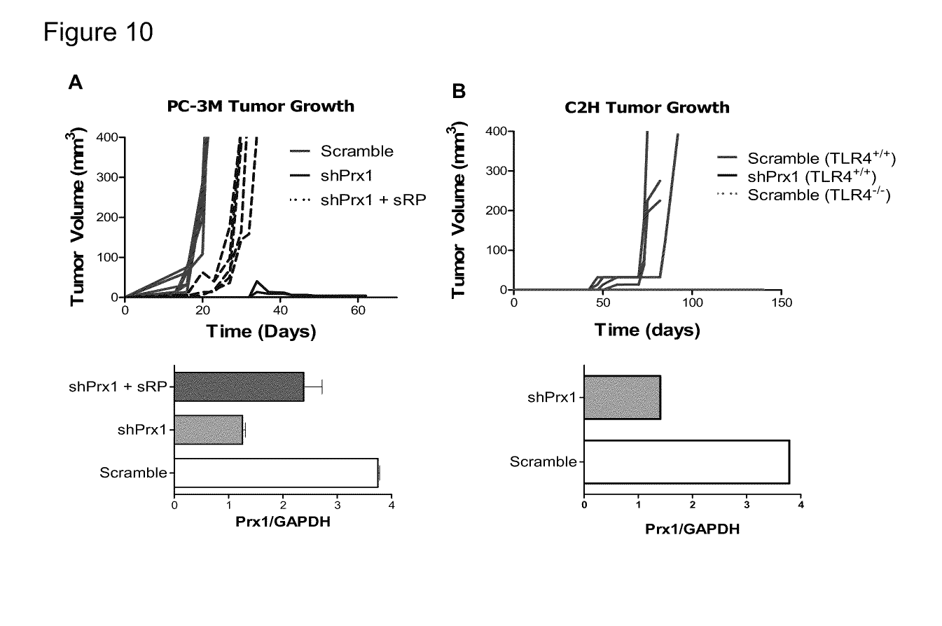

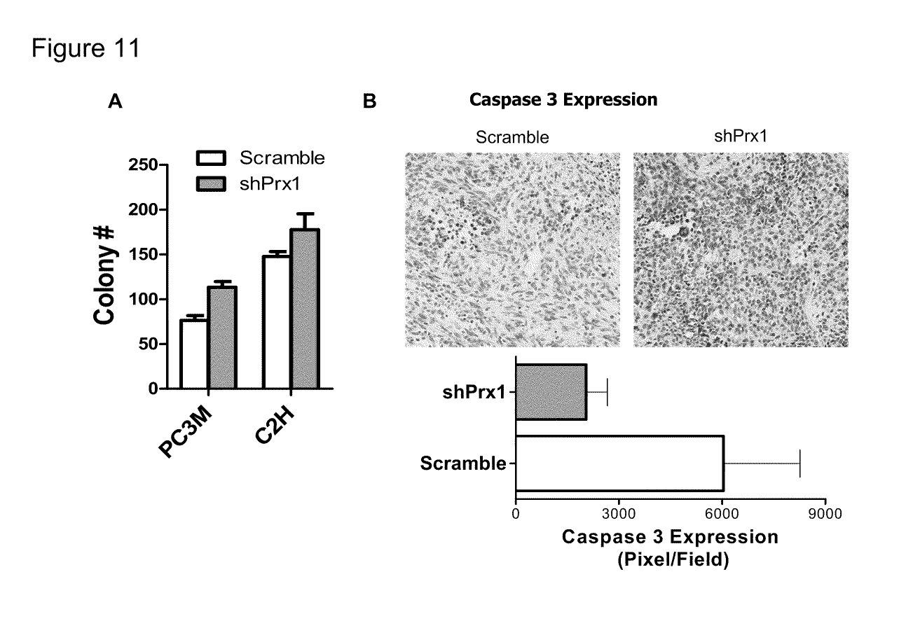

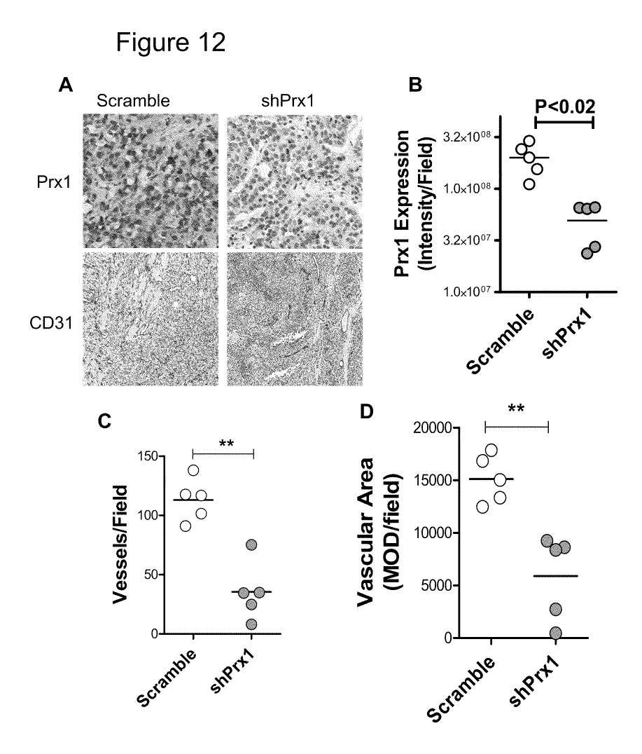

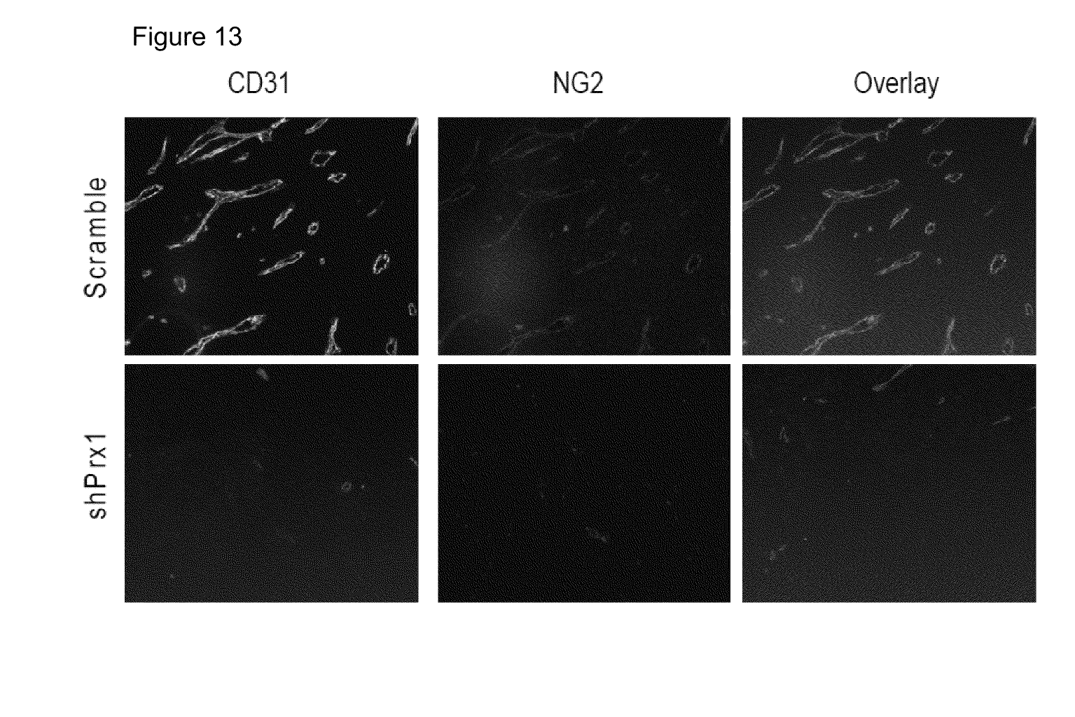

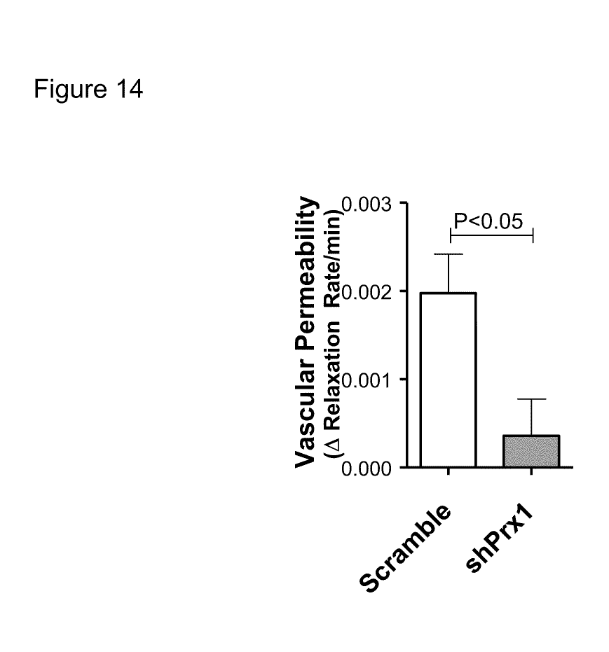

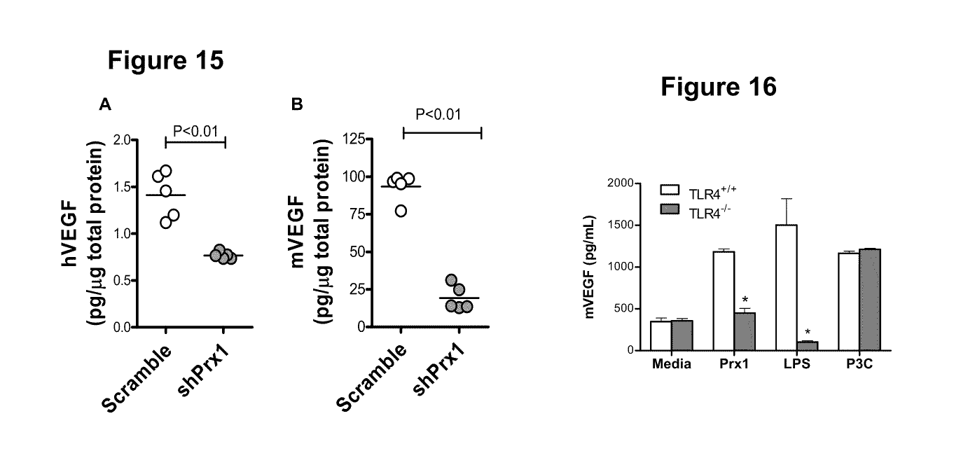

We have shown that Prx1 expression is elevated in prostate cancer (CaP) and that expression increases as the disease progresses (FIG. 9). The role of elevated Prx1 in tumors is unclear; however we have recently shown reduction of Prx1 levels by expression of shRNA specific for Prx1 results in inhibition of prostate tumor growth in two murine tumor models of CaP (FIG. 10). The loss of Prx1 has no effect on tumor cell growth in vitro or cell survival in vivo (FIG. 11). Examination of the tumors revealed that Prx1 expression correlated with the presence of tumor vessels (FIG. 12); in the absence of Prx1, the number of vessels was significantly reduced and less mature as measured by the extent of pericyte coverage (FIG. 13). Furthermore, the vessels that were present in tumors with reduced Prx1 levels were less functional. i.e., they had an increase in permeability (FIG. 14). Angiogenesis is regulated by a number of growth factors, including vascular endothelial growth factor (VEGF). Inhibition of Prx1 expression leads to a loss of VEGF expression within the tumor microenvironment (FIGS. 15 and 16).

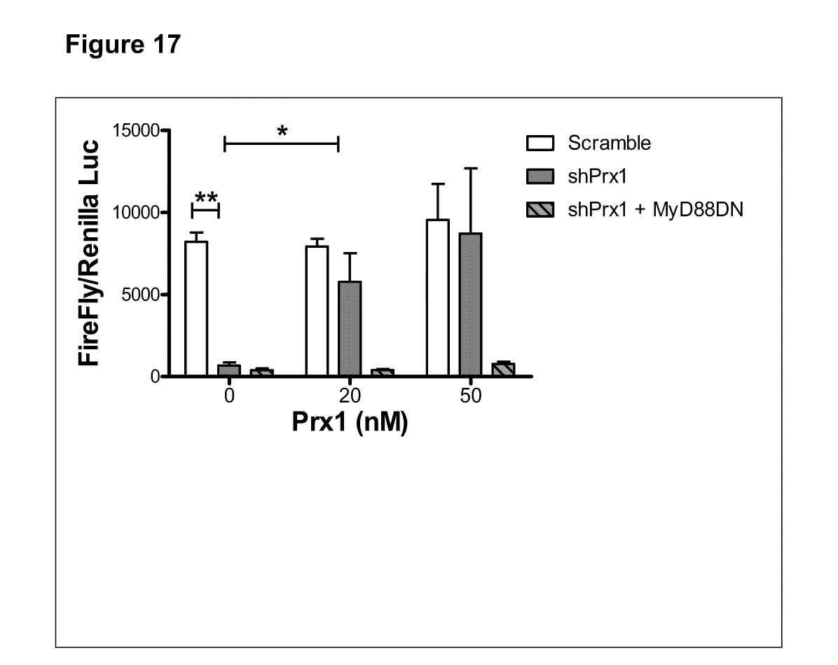

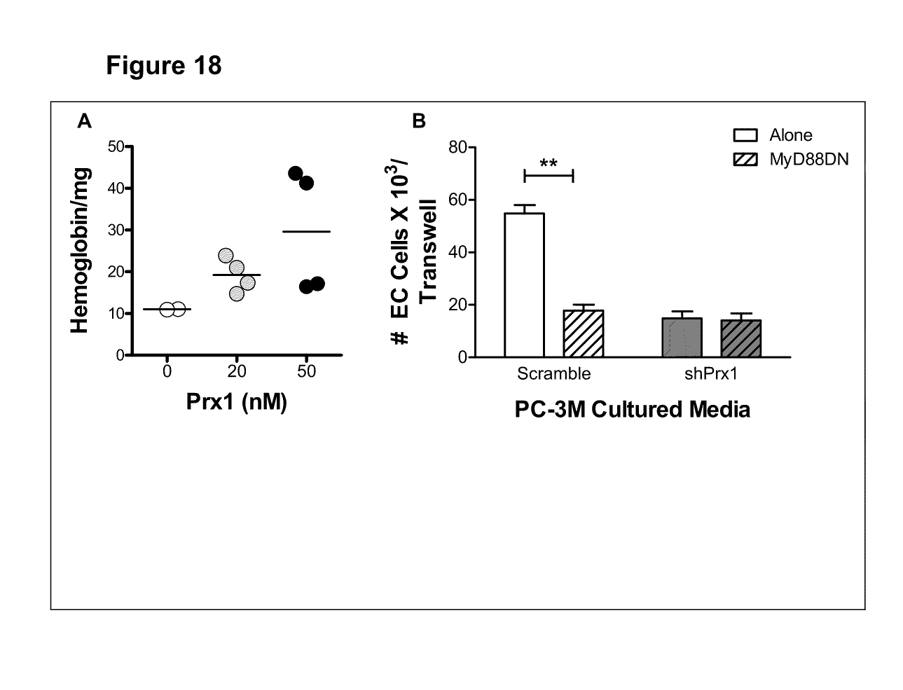

Recent studies have demonstrated that Prx1 can be secreted by non-small cell lung cancer cells, possibly via a non-classical secretory pathway. The function of extracellular/secreted Prx1 is unknown; however we have recently shown that secreted Prx1 binds to toll-like receptor 4 (TLR4) and stimulates the release of VEGF (FIG. 17). Furthermore Prx1 stimulates VEGF promoter activity (FIG. 17) and this stimulation is dependent upon TLR4 signaling.

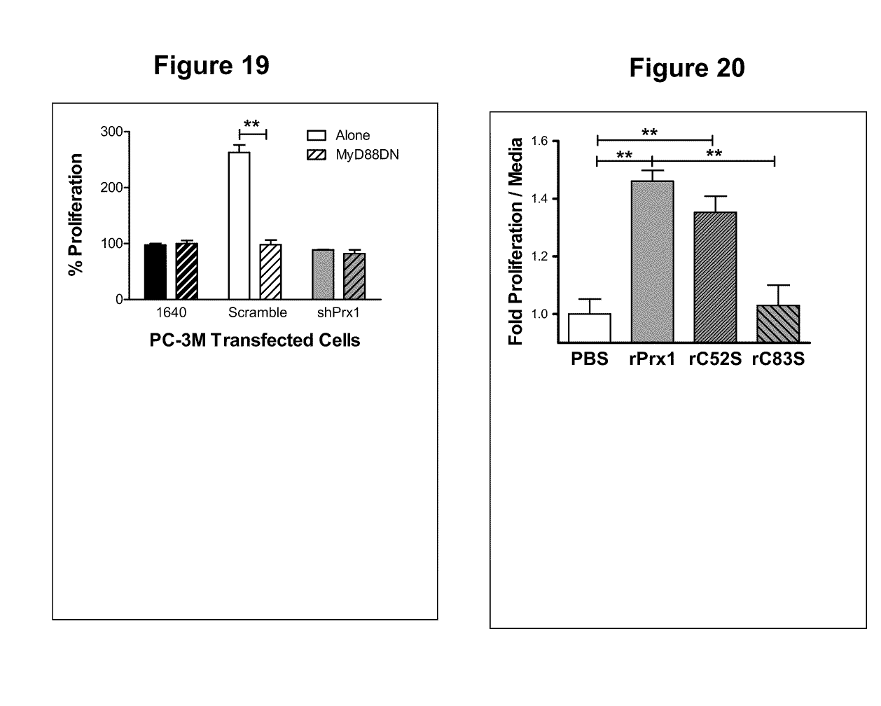

Angiogenesis and formation of new vessels is due in part to proliferation and migration of endothelial cells. Prx1 stimulates migration of endothelial cells in vivo and in vitro and the stimulation of migration is dependent upon TLR4 (FIG. 19). Prx1 also stimulates proliferation of endothelial cells in a TLR4 dependent manner (FIG. 19).

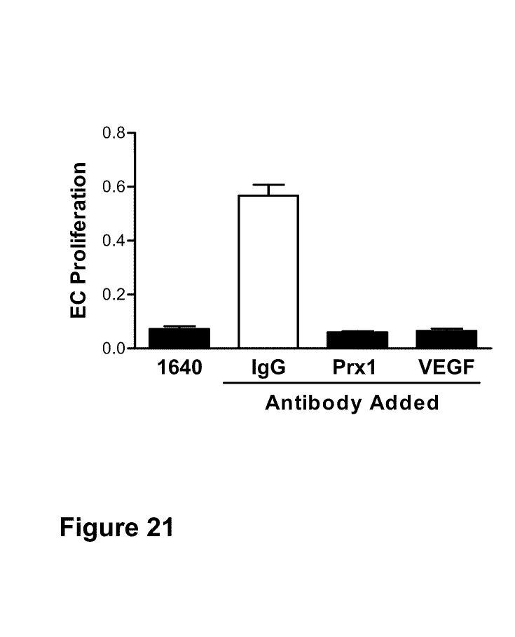

The ability of Prx1 to bind to TLR4 is dependent upon it chaperone activity (FIG. 20); Prx1 mutants that lack chaperone activity can not stimulate endothelial cell proliferation. Furthermore tumor cells that express Prx1 are unable to grow in mice that lack TLR4 (FIG. 9). We predict that inhibition of Prx1 or Prx1 chaperone activity will prevent activation of TLR4, block tumor angiogenesis and result in prevention of tumor growth. Inhibition can be achieved by shRNA specific for Prx1, inhibition of chaperone activity or antibodies specific for Prx1 (FIG. 21).

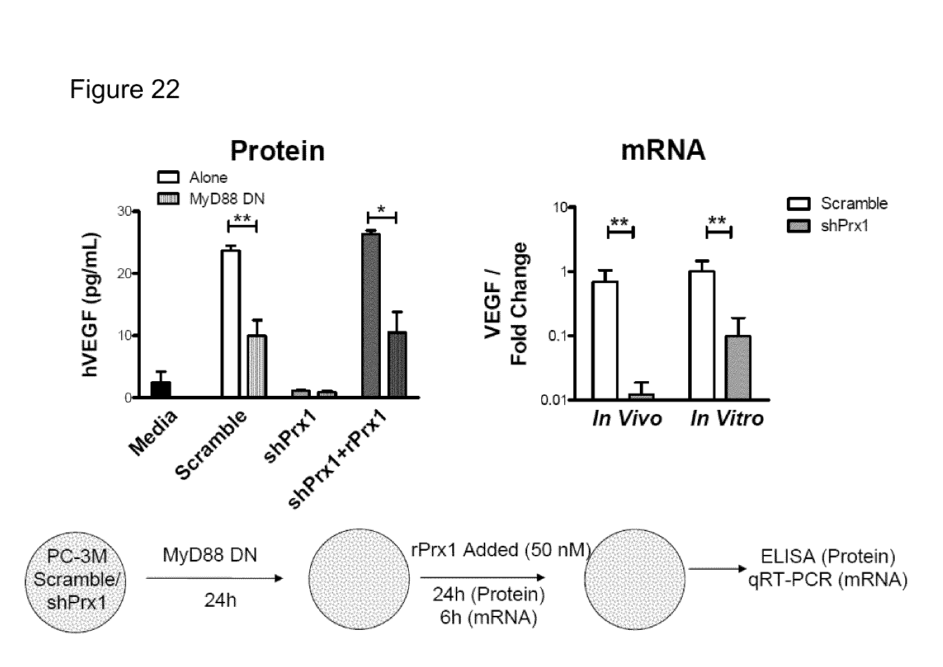

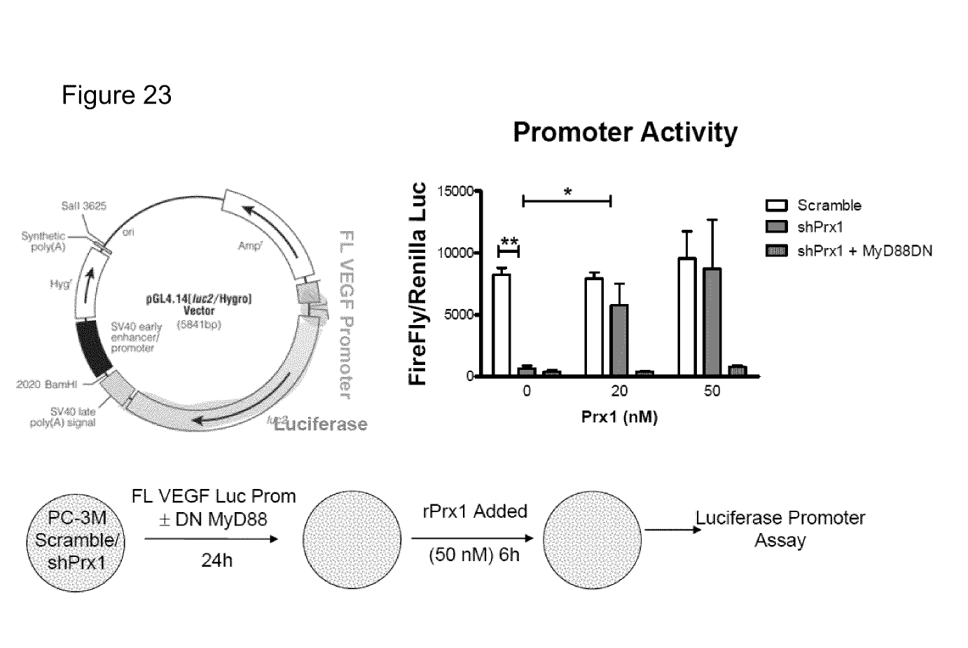

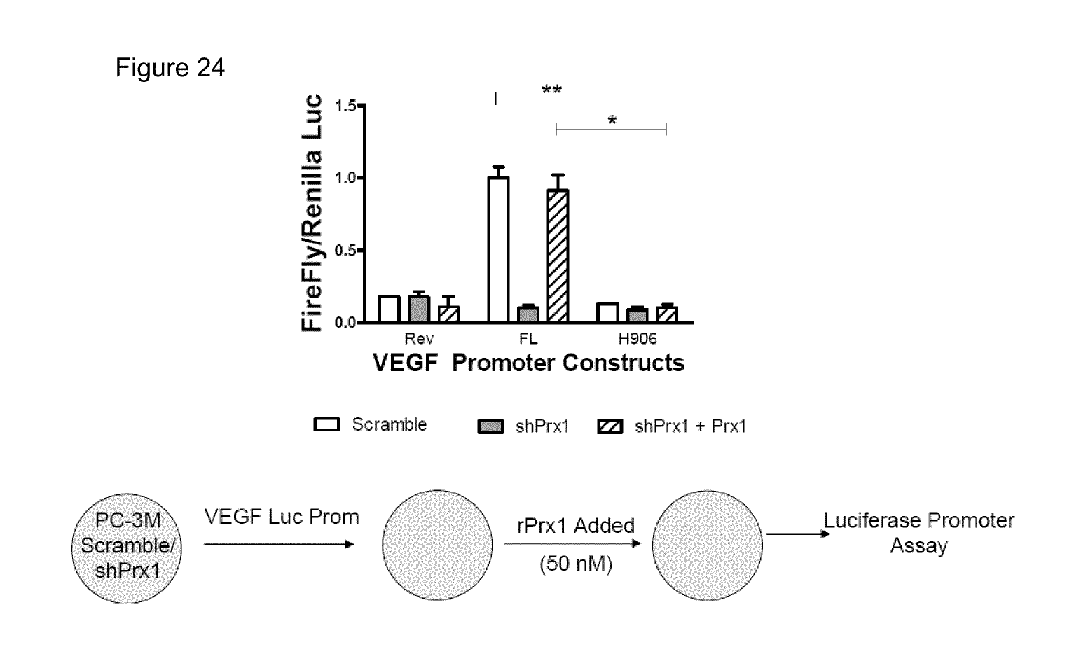

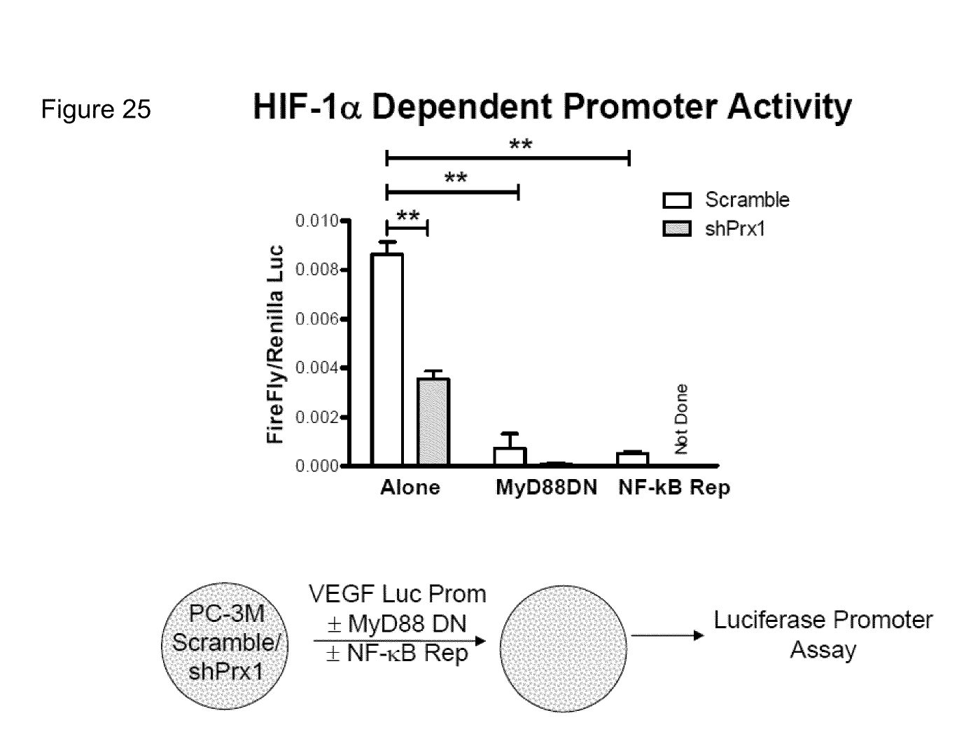

The information presented in FIGS. 22-25 further supports our discovery that Prx1 stimulates expression of VEGF mRNA and protein, and in particular that Prx1 stimulation of VEGF mRNA is regulated by the transcription factor HIF-1α and is dependent upon its interaction with TLR4, and that Prx1 stimulation of HIF-1α activity is dependent upon NF-κB activation of HIF-1α. Thus, it will be recognized from the foregoing that one advantage of the invention is that blocking TLR4 occurs upstream of VEGF induction. Another advantage is that Prx1 is found primarily within the tumor microenvironment, thus this therapy has the potential of having greater anti-angionenic tumor specificity and fewer side effects.

We claim:

1. A method for inhibiting angiogenesis in a tumor comprising administering to an individual a composition comprising an agent capable of inhibiting binding of peroxiredoxin 1 (Prx1) to Toll like receptor 4 (TLR4) such that angiogenesis in the tumor is inhibited subsequent to the administration.

2. The method of claim 1, wherein the agent is an antibody that can specifically recognize Prx1, or is a fragment of the antibody wherein the fragment can specifically recognize Prx1.

3. The method of claim 2, wherein the antibody is a monoclonal antibody.

4. The method of claim 1, wherein the agent is a fragment of Prx1.

5. The method of claim 1, wherein the individual is in need of treatment for a tumor selected from prostate, thyroid, lung, bladder breast and oral cancer tumors.

4. The method of claim 1, wherein the individual is in need of treatment for a prostate tumor.

5. The method of claim 1, wherein the inhibiting of the angiogenesis comprises a reduction in number of blood vessels in the tumor.

6. The method of claim 1, wherein the inhibiting of the angiogenesis comprises an increase in permeability of blood vessels in the tumor.

7. The method of claim 1, wherein the inhibiting of the angiogenesis is correlated with a reduction in vascular endothelial growth factor (VEGF) mRNA, VEGF protein, or a combination thereof in the tumor.
